(19) United States
(12) Patent Application Publication
(10) Pub. No.: US 2026/0363593 A1
(43) Pub. Date: Nov. 19, 2026

(54) SYSTEM AND METHOD FOR EXTERNAL INCONTINENCE MONITORING

(71) Applicant: Senior Shield Technologies, Inc., Woodstock, GA (US)

(72) Inventors: Kareem Naser Elfoulie, Lawrenceville, GA (US); Hyungiun Park, Woodstock, GA (US); Rossy Thien Nhan Dang, Lawrenceville, GA (US); Mingshuang Wang, Philadelphia, PA (US); Nahid Hossain, Lawrenceville, GA (US)

(21) Appl. No.: 19/649,816

(22) Filed: Apr. 16, 2026

Related U.S. Application Data
(60) Provisional application No. 63/790,616, filed on Apr. 17, 2025.

(51) Int. Cl. A61B 5/20; A61B 5/00

(52) U.S. Cl. CPC A61B 5/202; A61B 5/6804; A61B 5/7282; A61B 2562/0219; A61B 2562/0271

(57) ABSTRACT

A system for monitoring incontinence, the system including a garment wearable by a user, the garment comprising an absorbent region, a selectively openable enclosure defining a cavity, the enclosure configured to adhere to the garment proximate the absorbent region and along a sagittal plane of the user, a sensing array positioned within the cavity of the enclosure, the sensing array having a first end proximate an anterior surface of the user and a second end proximate a posterior surface of the user, the sensing array including a plurality of temperature sensors arranged linearly between the first end and the second end, at least one impedance sensing region, at least one motion sensor positioned proximate the first end or the second end and at least one gas sensor positioned proximate the first end or the second end.

### CROSS REFERENCE TO RELATED APPLICATIONS

This application claims the benefit of priority to US Provisional Application Ser. No. 63/790,616, filed Apr. 17, 2025, the entire contents of which are hereby incorporated by reference herein.

### BACKGROUND

Incontinence is a medical condition in which a patient loses control of their bladder or bowels. This condition affects a significant portion of the elderly population, with rates in long-term care facilities reaching over 70%. Patients with incontinence often wear garments, such as briefs, to contain excrement. Soiled garments must be changed quickly, as prolonged exposure to urine or feces can cause harmful complications, including urinary tract infections, sepsis, pressure sores, and dermatitis. An urgent care crisis exists due to a growing elderly population and a shortage of nurses and caregivers, resulting in many patients sitting soiled for extended periods. Currently, caregivers must physically probe to check whether a garment is soiled, which is uncomfortable, unsanitary, and undignified.

Available incontinence monitors may use disposable moisture sensors to check for urine. Such products are often not reusable and may be invasive, placing sensors near sensitive skin and organs. Studies on these products report varying accuracy for moisture detection. Embedded moisture sensors in garments may also limit patients to specific brands rather than being brand-agnostic. Although urinary incontinence is more prevalent, both urinary and fecal incontinence see higher incidence rates in older populations, with fecal incontinence being the second leading cause of nursing home enrollment. Direct moisture sensing may missfecal incontinence events, as one manufacturer noted that feces could interfere with registration of urine by blocking the sensors.

Some care facilities practice scheduled checks of patients for incontinence on regular time intervals. However, such scheduled checks may be ineffective because incontinence events can happen at any time and care facilities often experience understaffing.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a system for monitoring incontinence, the system comprising: a garment wearable by a user, the garment comprising an absorbent region; a selectively openable enclosure defining a cavity, the enclosure configured to adhere to the garment proximate the absorbent region and along a sagittal plane of the user; a sensing array positioned within the cavity of the enclosure, the sensing array having a first end proximate an anterior surface of the user and a second end proximate a posterior surface of the user, the sensing array comprising: a plurality of temperature sensors arranged linearly between the first end and the second end; at least one impedance sensing region; at least one motion sensor positioned proximate the first end or the second end; and at least one gas sensor positioned proximate the first end or the second end.

In some embodiments, the system includes an overlay element arranged to contact at least a portion of the plurality of temperature sensors, the overlay element comprising a conductive material.

In some embodiments, the conductive material comprises at least one of copper or silver.

In some embodiments, the at least one impedance sensing region comprises a first capacitive sensing region positioned proximate the first end and a second capacitive sensing region positioned proximate the second end of the sensing array.

In some embodiments, the at least one gas sensor comprises at least a first gas sensor proximate the first end and at least a second gas sensor proximate the second end.

In some embodiments, the first and second gas sensors are each configured to detect different gaseous compounds associated with urination and defecation, respectively.

In some embodiments, the first gas sensor is configured to detect at least one of ammonia (NH3), dihydrogen sulfide (H2S), odor or volatile organic compounds (VOC); and the second gas sensor is configured to detect at least one of methane (CH4), dihydrogen sulfide (H2S), odor or volatile organic compounds (VOC).

In some embodiments, the at least one motion sensor comprises an inertial measurement unit (IMU), the IMU comprising at least one of an accelerometer, a gyroscope or a magnetometer.

In some embodiments, the sensing array is communicatively coupled to at least one processor.

In some embodiments, the enclosure comprises a coupling element extending longitudinally along an exterior surface, each end of the coupling element extending beyond the ends of the enclosure, the ends of the coupling element configured to couple to the garment.

The herein disclosed subject matter is also directed to a method for monitoring incontinence, the method comprising: positioning a sensing array along an absorbent region of a garment, the sensing array arranged substantially along an axis aligned with a sagittal plane of a user wearing the garment, the sensing array comprising: a first end proximate an anterior surface of the user and a second end proximate a posterior surface of the user; a plurality of temperature sensors arranged coaxially with the axis between the first end and the second end; at least one impedance sensing region; at least one gas sensor; and at least one motion sensor; detecting, via the sensing array, an adhesion state of the sensing array to the garment; detecting, via the sensing array, an incontinence event within the garment; and transmitting at least one signal from the sensing array to at least one processor in communication with the sensing array.

In some embodiments, the method further includes retrieving an incontinence event history associated with the user prior to the positioning of the sensing array.

In some embodiments, the method further includes generating, based on the adhesion state and the incontinence event, an incontinence diary associated with the user.

In some embodiments, detecting the adhesion state comprises detecting at least one of a motion datum via the at least one motion sensor or a capacitive datum via the at least one impedance sensing region.

In some embodiments, detecting the incontinence event comprises detecting at least one of an excreta presence, an excreta location, or an excreta volume within the garment.

In some embodiments, detecting the excreta location comprises at least one of detecting a gaseous compound via the at least one gas sensor or detecting a temperature gradient across the plurality of temperature sensors.

In some embodiments, detecting the excreta volume comprises detecting a change in capacitance via the at least one impedance sensing region.

In some embodiments, detecting the excreta presence comprises detecting a temperature at at least one temperature sensor above a predetermined temperature threshold associated with a clean garment.

In some embodiments, the method further includes training a machine learning model on at least the incontinence event history associated with the user; and executing, by the at least one processor, the machine learning model, to classify the signals for each sensor of the sensing array.

In some embodiments, the machine learning model is at least one of a convolutional neural network (CNN), recurrent neural network (RNN) or a long short-term memory network.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description. It is to be understood that both the foregoing general description and the following detailed description are exemplary and are intended to provide further explanation of the disclosed subject matter claimed.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

Furthermore, the drawings may contain text or captions that may explain certain embodiments of the present disclosure. This text is included for illustrative, non-limiting, explanatory purposes of certain embodiments detailed in the present disclosure.

FIG. 1 is a perspective view of a system for external incontinence monitoring placed on an absorbent region of a garment in accordance with aspects of the present disclosure.

FIG. 2 is a side view of adhesion of the system for external incontinence monitoring to a person's garment in accordance with aspects of the present disclosure.

FIG. 3A is a top view of an enclosure for carrying a sensing array of the incontinence monitoring system in accordance with aspects of the present disclosure.

FIG. 3B is a perspective schematic view of a sensing array within a layered enclosure in accordance with aspects of the present disclosure.

FIG. 4A is a top view of an embodiment of a sensing array layout in accordance with aspects of the present disclosure.

FIG. 4B is a planform schematic view of an arrangement of temperature sensors of a sensing array according to aspects of the present disclosure.

FIG. 5A is another schematic representation of a sensing array in accordance with aspects of the present disclosure.

FIG. 5B is a representation of a plurality of temperature sensors including individual overlay elements in accordance with aspects of the present disclosure.

FIG. 6 is a plot of temperature readouts associated with a plurality of temperature sensors of the sensing array in accordance with aspects of the present disclosure.

FIG. 7 is a simulation of thermal conductance of an overlay element in accordance with aspects of the present disclosure.

FIG. 8 is a plot of capacitive readings of sensors during a urinary incontinence event in accordance with aspects of the present disclosure.

FIG. 9 is a schematic layout of impedance sensing regions on an absorbent region of a garment in accordance with aspects of the present disclosure.

FIG. 10 is a plot of capacitance associated with excreta volume in accordance with aspects of the present disclosure.

FIG. 11 is a linear regression of capacitance associated with excreta volume in accordance with aspects of the present disclosure.

FIG. 12 is a plot of capacitance associated with an incontinence event in accordance with aspects of the present disclosure.

FIG. 13 is a plot of gas resistance detected by digital gas sensors during urinary and fecal incontinence events in accordance with aspects of the present disclosure.

FIG. 14 is a plot of analog gas detection associated with specified chemical compounds during urinary and fecal incontinence events in accordance with aspects of the present disclosure.

FIG. 15 is a plot of relative ammonia levels associated with a urinary incontinence event in accordance with aspects of the present disclosure.

FIG. 16A is a representation of an embodiment of a sensing array and selectably openable enclosure for detecting fecal incontinence events in accordance with aspects of the present disclosure.

FIG. 16B is a representation of an embodiment of a selectably openable enclosure with labeling in accordance with aspects of the present disclosure.

FIG. 17 is an exemplary flowchart of a method for detecting incontinence events and adhesion states associated with the sensing array in accordance with aspects of the present disclosure.

FIG. 18A is a detailed flowchart of a method for using an incontinence monitoring system in accordance with aspects of the present disclosure.

FIG. 18B is a continuation of FIG. 18A of a detailed flowchart of a method for using an incontinence monitoring system in accordance with aspects of the present disclosure.

FIG. 18C is a continuation of FIGS. 18A-18B of a detailed flowchart of a method for using an incontinence monitoring system in accordance with aspects of the present disclosure.

FIG. 18D is a continuation of FIGS. 18A-18C of a detailed flowchart of a method for using an incontinence monitoring system in accordance with aspects of the present disclosure.

FIG. 19A is a schematic representation of machine learning models for incontinence and adhesion state detection in accordance with aspects of the present disclosure.

FIG. 19B is a continuation of FIG. 19A of a schematic representation of machine learning models for incontinence and adhesion state detection in accordance with aspects of the present disclosure.

FIG. 20A is a schematic representation of an incontinence monitoring system with selectably engageable fastening elements in accordance with aspects of the present disclosure.

FIG. 20B is a schematic representation of an incontinence monitoring system with selectably engageable fastening elements in accordance with aspects of the present disclosure.

FIG. 20C is a schematic representation of an incontinence monitoring system with selectably engageable fastening elements in accordance with aspects of the present disclosure.

FIG. 21 is a schematic representation of an incontinence monitoring system with a fan shaped planform in accordance with aspects of the present disclosure.

FIG. 22 is a schematic representation of an incontinence monitoring system in accordance with aspects of the present disclosure.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention.

Furthermore, it is important to note that, as used herein, “a” and “an” each generally denotes “at least one” but does not exclude a plurality unless the contextual use dictates otherwise. When used herein to join a list of items, “or” denotes “at least one of the items” but does not exclude a plurality of items of the list.

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure.

Reference will now be made in detail to exemplary embodiments of the disclosed subject matter, an example of which is illustrated in the accompanying drawings. The method and corresponding steps of the disclosed subject matter will be described in conjunction with the detailed description of the system.

The present disclosure is directed to various embodiments of a system and method for an incontinence monitoring pad with an electronic sensing array and a fabric sleeve to detect urine or feces within a garment without skin contact, primarily worn by patients facing incontinence, babies, children dealing with enuresis, bedwetting, or toilet (or ‘potty’) training, or animals wearing diapers.

For the purposes of this disclosure, the term “pad” refers to the combination of an electronic sensing array and a selectably openable enclosure in which the sensing array may be embedded or placed. For the purposes of this disclosure, the term “garment” refers to a brief, diaper, pullup, underwear or similar article of clothing characterized by having some region thereof intended to absorb human excreta. For the purposes of this disclosure, the term “absorbent region” refers to a portion of the garment that interfaces with human excreta and may contain material that is hygroscopic. For the purposes of this disclosure, the term “interior” when referring to “interior of the garment” refers to the portion of the garment facing the wearer when worn, especially the portions of the garment exposed to human orifices when worn. For the purposes of this disclosure, the term “exterior” of the garment refers to the portion of the garment which faces away from the wearer when worn.

The present disclosure is directed to various embodiments of systems and methods for monitoring incontinence in a patient via an incontinence monitoring system, which may include a sensing array placed within an enclosure, such as a fabric sleeve and communicating a patient's incontinence status or device adhesion status to a phone, tablet, laptop, desktop computer, or other electronic device via wired or wireless protocols via a user interface.

In various embodiments, the sensing arrays described herein may be placed on an absorbent region of a user-wearable garment. In various embodiments, the sensing array may exceed the length of the absorbent region of the garment without exceeding the overall length of said garment. Various embodiments of the sensing array may have a width that is narrower than the width of the absorbent region of said garment, which may be advantageous by reducing bulk sizing and material costs while also avoiding direct contact with a person's skin. In various embodiments, the length of the sensing array may be equivalent to or less than the length of the absorbent region of a garment. In some embodiments, the width of the sensing array may be equal to or greater than the width of the absorbent region of the garment to profile the entire garment.

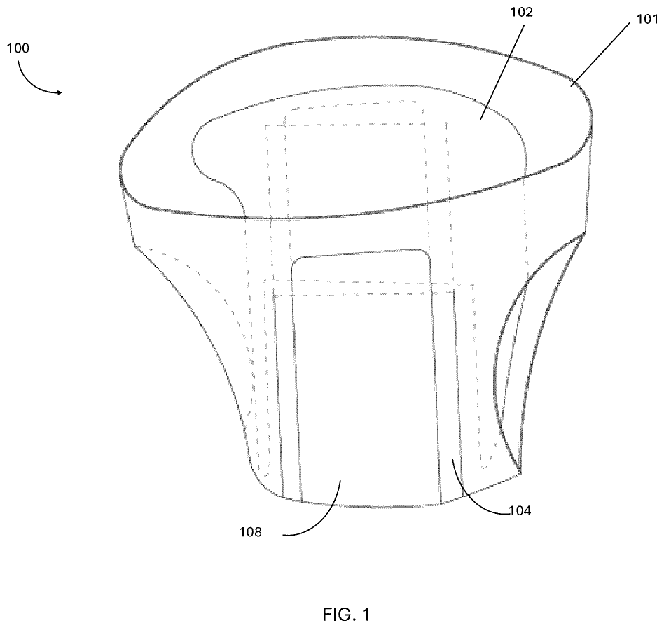

Referring now to FIG. 1, a perspective view of a system 100 for incontinence monitoring is shown. The system 100 may include a garment 101 intended to be worn by a patient, user, child, adult or animal around the excrement-expelling orifices thereof. In other words, the garment 101 may be wearable around the waist of a human patient such that it extends about the buttocks, groin and between the legs proximate the human genitals and along the perineum. In various embodiments, the garment 101 may include a front or anterior section overlaying the anterior surface of the human body proximate the urethra in a male (and extending inferiorly towards the area of the female urethra). In various embodiments, the garment 101 may extend along the sagittal plane between the legs such that it overlays the buttocks of the wearer proximate the anus. As shown in FIG. 1, the garment 101 may be embodied as a brief or underwear, including an upper portion formed as a continuous waistband and extending downward in a concave shape correspondingly shaped to the area of the human body. As shown, the garment 101 may include two lateral openings such that the user or medical provider can insert the wearer's legs therethrough to pull up the garment 101 into position such that the garment covers the urethra and the anus of the wearer. In various embodiments the garment may be disposable, such as in the case of a diaper or depend, or washable and reusable, such as underwear.

With continued reference to FIG. 1, garment 101 may include an absorbent region 102. Absorbent region 102 may be a portion of the garment closest to the urethra and anus of the user. For example, the absorbent region 102 may extend from an anterior point proximate the waistband, along the sagittal plane between the legs of the patient and terminating at a posterior point proximate the waistband at the back of the user. In various embodiments, the absorbent region 102 may be substantially rectangular in perimeter, and compliantly arc along with garment 101 to complement the legs, genitals and buttocks of the wearer of the garment 101. In various embodiments, the absorbent region may be fan-shaped or of variable width along its length in order to adequately cover the area of garment 101 closest to the urethra and the anus of the wearer, an example fan-shape is shown in FIG. 21.

In various embodiments, the absorbent region 102 may be attached to the interior of the garment. In various embodiments, the absorbent region 102 may be embedded within the garment 101. In various embodiments, the absorbent region may be integral with the garment 101. In various embodiments, the absorbent region 102 may be made from a hygroscopic material such that the absorbent material 102 absorbs a greater volume of fluid or excreta than does the rest of garment 101.

With continued reference to FIG. 1, system 100 may include a selectively openable enclosure 104. Enclosure 104 may be adhered to the exterior of the garment 101 substantially corresponding to the absorbent area 102 on the interior of the garment 101.

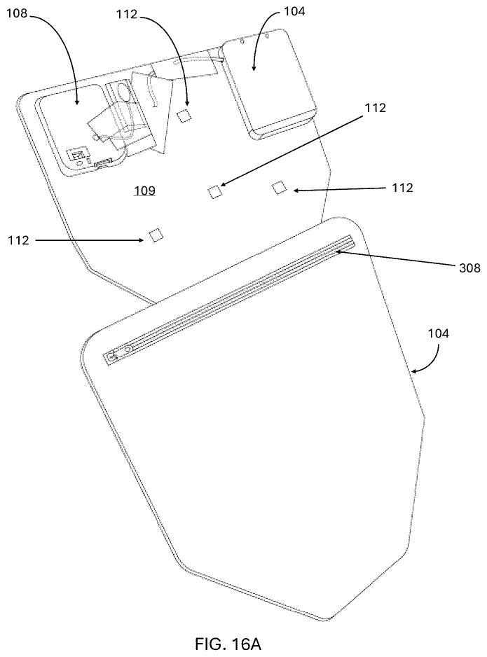

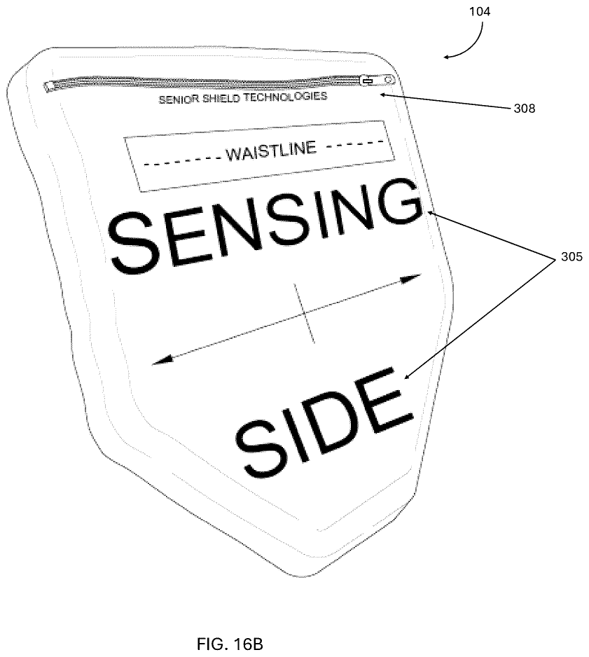

Enclosure 104 may be an elongate body configured to adhere to the garment 101 along the sagittal plane of the wearer when the garment 101 is positioned on the body. That is to say, enclosure 104 may extend from an anterior surface of the garment 101, between the legs of the user and up along a posterior surface of the garment 101. In various embodiments, enclosure 104 is placed such that human excreta expelled from the body into the garment 101 and absorbed by absorbent region 102 is proximate the enclosure 104, separated by the fabric of the exterior of the garment 101. In various embodiments, enclosure 104 may have a substantially rectilinear planform shape, with the two shorter sides at the anterior and posterior-most positions, and the relatively longer sides parallel to the sagittal plane of the wearer. In various other embodiments, enclosure 104 may be shaped correspondingly to other portions of the human or animal body on which it is placed. As shown in FIGS. 16A-16B, enclosure 104 may be trapezoidal in shape such that it covers a wider area of the human buttocks proximate the anus.

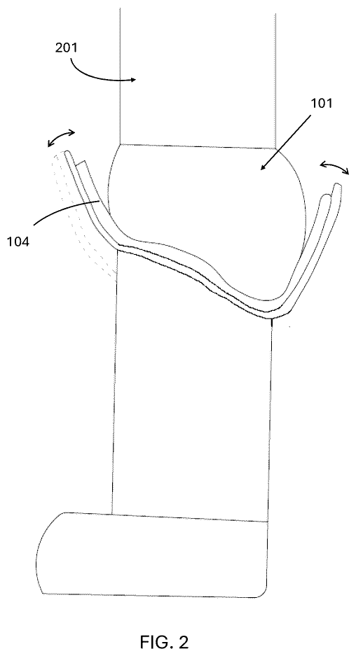

In various embodiments, enclosure 104 may be attached or adhered to the exterior of garment 101 while garment 101 is already worn by the user, as shown in FIG. 2. FIG. 2 shows a sagittal section view of a schematic human 201. Human 201 is shown facing leftward with their right leg extending downward. The garment 101 is shown already worn by the human 201. Garment 101 is shown extending at its highest point along the waist of the human 201 and overlaying the groin proximate the anterior surface (lefthand side of page) and overlaying the buttocks proximate the posterior surface (righthand side page). As shown in FIG. 2, the enclosure 104 is placed along the sagittal plane of the garment 101 from the front, between the legs and along the backside, as denoted by the curved arrows.

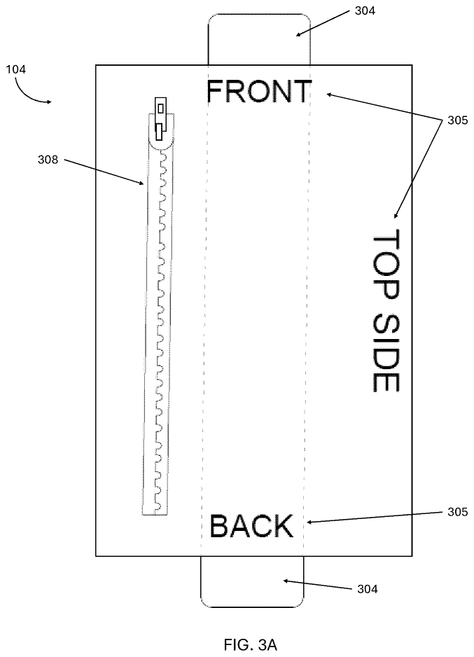

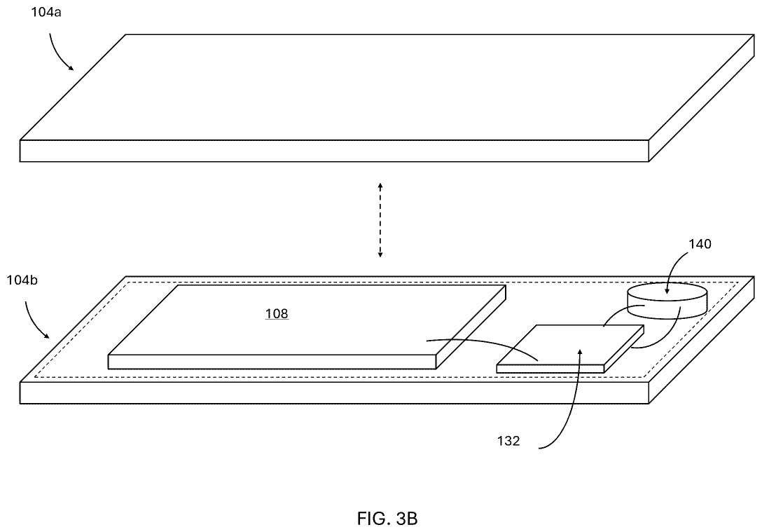

Referring now to FIG. 3A, a planform view of an embodiment of enclosure 104 is shown. As previously discussed, although enclosure 104 is shown for simplicity here as a rectilinear planform perimeter, other embodiments may be irregularly shaped or shaped to accommodate one or more the sensing array (to be described below) or the human/animal user of the system 100. Enclosure 104 may define an interior cavity in which a sensing array (108) may be inserted. Enclosure 104 may encapsulate the sensing array 108 in order to restore dignity to patients by keeping sensors away from skin and sensitive organs.

In various embodiments, enclosure 104 may be formed from a compliant, flexible material that conforms to the shape of garment 101 for secure adherence. For example, enclosure 104 may be formed from one or more fabric layers attached proximate their respective perimeters to define the interior cavity. In various embodiments, a fabric enclosure 104 may carry the sensing array 108 within the interior cavity and adhered to garment 101. For example, and without limitation, a first layer of fabric may be used to form an upper portion the enclosure 104, a second layer of fabric may be placed underneath the first layer and may then be attached along the overlapping perimeters of each layer of fabric, forming a cavity between the two layers of fabric that can therefore support the sensing array 108. A schematic example of this plied enclosure is shown in FIG. 3B, where the upper layer is 104a and the lower layer is 104b. The characteristic shape, perimeter, shadow, or outline of the layers of fabric may resemble those of the sensing array 108 and/or its substrate.

As shown in FIG. 3A, enclosure 104 may include lettering, icons, symbols, or graphics (collectively, labeling 305) placed onto the exterior of the enclosure 104 to designate different regions of the enclosure 104. Labeling 305 may guide a user, patient, or medical provider in orienting said sensing array 108 correctly into said enclosure 104.

With continued reference to FIG. 3A, enclosure 104 may include a selectively closable opening 308 formed through at least one layer thereof in order to gain access to the interior cavity of the enclosure 104. In various embodiments, a cutout may be made into one or more layers of fabric that form the walls of enclosure 104 to embed a fastener or a closure for the cavity via surface adhesion or sewing. In various other embodiments, a fastener mechanism can be attached onto either face of either layer of fabric via surface adhesion or sewing. In various embodiments, surface adhesion may include the use of adhesive, tape, or another substance with adhesive compound. In various embodiments, surface adhesion may also include a heat transfer method for bonding surfaces together. In various embodiments, opening 308 may be secured via mechanical fastening methods, such as a zipper, a hook-and-loop fastener, a pair of male and female snap-fit connectors, or a button that slips into a buttonhole (or slot). The use of such fasteners may facilitate a reusable system 100 for incontinence monitoring by enabling a sensing array to be inserted into and removed from a sleeve.

In various embodiments, enclosure 104 may be reinforced by the inclusion of multiple layers in lamination. For example, and without limitation, enclosure 104 may include additional layers of fabric adhered underneath the bottommost layer of the aforementioned layers of fabric. Within said additional layers, padding, foam, or fill material may be included to promote a softer feel when worn by a patient.

In various embodiments, enclosure 104 may be attached or adhered to garment 101 via an adhesive sheet to affix the perimeter of said enclosure 104 onto the exterior of said garment 101. In various embodiments, said adhesive sheet may be single-sided or double-sided adhesive. In various embodiments, adhesion may also be achieved by placing a double-sided adhesive sheet that overlaps with the enclosure 104 and resides between said enclosure 104 and the exterior of a garment 101 when attached together. In various embodiments, an adhesive sheet coextensive with the enclosure 104 may be placed between the enclosure 104 and the exterior of the garment 101 to attach them to one another. In various embodiments, mechanical fasteners may be utilized to adhere or attach the enclosure 104 to the garment 101. For example, clips, pins or hook-and-loop closures may be used to affix the enclosure 104 onto the exterior of garment 101.

Further, as shown in FIG. 3, enclosure 104 may include a coupling element 304 that extends beyond the ends of the enclosure 104 along the sagittal plane. That is to say, the coupling element 304 and enclosure 104 may include collinear center lines with the coupling element 304 relatively longer than the enclosure 104. For example, and without limitation, the coupling element 304 may be a fabric elastic band placed on the exterior of the bottom-most layer of enclosure 104, and with a length at rest extending beyond the overall length of said enclosure 104. In various embodiments, a disposable method of fastening the enclosure 104 to garment 101 could be achieved by placing a double-sided adhesive sheet onto one or both of each exposed end of coupling element 304 or the exterior of a garment, and then pressing the exposed surface of said adhesive sheet into each end the coupling element 304 into the garment exterior on each side. In various other embodiments, one or more mechanical fasteners may be placed onto each exposed end of the coupling element 304 with adhesive or be permanently connected to said ends of band. In various other embodiments, enclosure 104 mating connectors may be placed on both the exposed ends of the coupling element 304 and exterior of garment 101 which are connected to affix the enclosure 104 onto garment 101.

In various embodiments, enclosure 104 may be formed from a fabric material. Said fabric material may be one or more of waterproof or water-resistant. In some embodiments, seam sealing tape may be used to cover over sewn seams of the enclosure 104. In some embodiments, a protective coating or film may be applied to a fabric layer or multiple fabric layers to confer water resistance or waterproofing. In some embodiments, fabric material may be hypoallergenic. In some embodiments, fabric material may be latex-free. In some embodiments, fabric material may be certified for pediatric or geriatric use. In some embodiments, fabric material may be medical grade. In some embodiments, fabric material may be breathable.

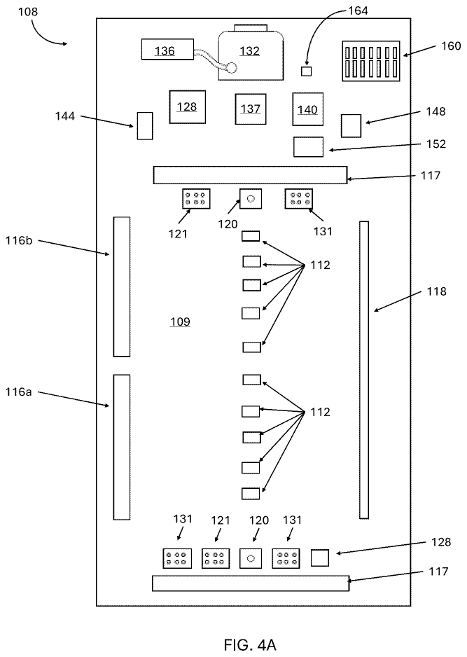

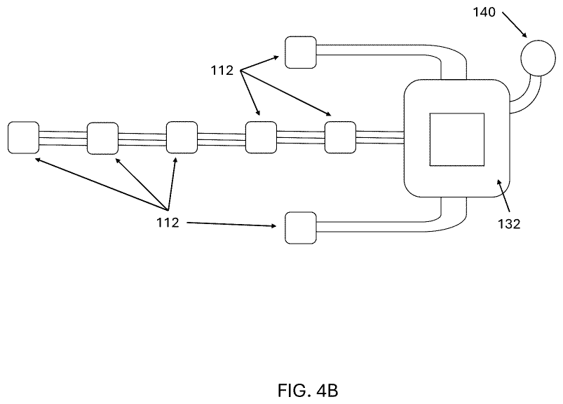

Referring now to FIG. 4A, a schematic view of a sensing array 108 of the incontinence monitoring system 100 is shown. Although shown on a rectangular substrate 109, one of skill in the art would appreciate that the components described below may be employed on one or more other form factors, such as printed circuit boards (PCB), flexible PCBs, sensors embedded in a substrate and communicatively connected to one more integrated circuits (IC) or embedded directly within enclosure 104 or garment 101 as described above. Further, sensing array 108 may be inserted into enclosure 104 for adherence to garment 101 to monitor one or more of adhesion states and incontinence events experienced by the user. Accordingly, one of skill in the art would appreciate that the illustrative embodiments shown and described herein are to explain the spirit of the system, but other arrangements, including relative arrangement and spacing between the components is within the scope of this disclosure.

As discussed above, sensing array 108 may be formed with a substantially rectilinear planform shape. Due to the intended positioning of the sensing array 108 proximate the urethra and anus of a human user along the sagittal plane, reference will be made to the ‘front’ of the sensing array, which corresponds to the anterior most position thereof. Conversely, reference to the ‘back’of the sensing array refers to the posterior most position thereof.

In various embodiments, sensing array 108 may be a single-sided circuit board or a single-sided arrangement of electronic components atop conductive traces or wires. In various embodiments, the sensing array may be a double-sided circuit board or a double-sided arrangement of electronic components atop conductive traces or wires. In various embodiments, the sensing array may be a single layer circuit board. In some embodiments, the sensing array may be a multi-layer circuit board. In various embodiments, mechanical stiffeners may be placed underneath electronic components to reduce the probability of solder joint failure. In some embodiments, the sensing array may comprise common bus lines, hand-cut or machine cut, for transmitting power, ground, and data signals; there may be direct soldering of components onto said bus lines, or there may be soldering of wires between components and said bus lines. In various embodiments, electronic components may be directly wired to each other along common pins. In various embodiments, sensing array 108 may be formed by printing, deposition, or fabrication of sensing elements described herein onto a flexible substrate. In various embodiments, said substrate may be textile-based or fabric. Said deposited sensing elements may be achieved with additive polymer manufacturing; use of conductive threads or fabrics; or conductive fibers or pieces, which may be metals.

In various other embodiments, sensing array 108 may be formed by the placement of discrete sensing units onto garment 101. In various embodiments, said discrete units may be placed in a serial fashion. In various embodiments, said discrete units may be isolated from each other. In various embodiments, said discrete units may also collaborate with each other via the connection of each discrete unit to a neighbor via wires, cables, direct contact, physical connectors, or wireless connection methods. In various embodiments, said discrete sensing units may be placed individually. In various embodiments, said discrete sensing units may each have a CPU and capacity to receive and transmit data to a central database.

In various embodiments, the sensing array 108, or portions of said sensing array 108, may use water-resistant or waterproof substrate to encase the electric circuit, conductive traces, or wires. In various embodiments, the sensing array 108, or portions of said sensing array 108, may be embedded into a water-resistant or waterproof casing. For example, and without limitation, said casing may include electronic potting compound composed of, for example, silicone, epoxy, resin, polyurethane, or similar materials. In various embodiments, said casing may be or include a water-resistant or waterproof coating, such as a conformal coating. In various embodiments, said casing may be achieved by placing the sensing array 108 between multiple layers of waterproof or water-resistant material, such as fabric. In some embodiments, the sensing array 108, or portions of said sensing array 108, may be permanently sealed within multiple layers of waterproof or water-resistant material.

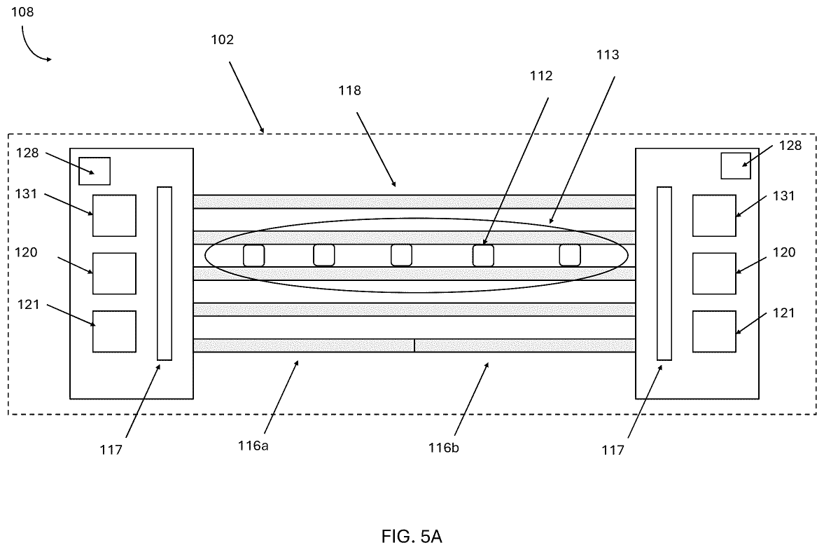

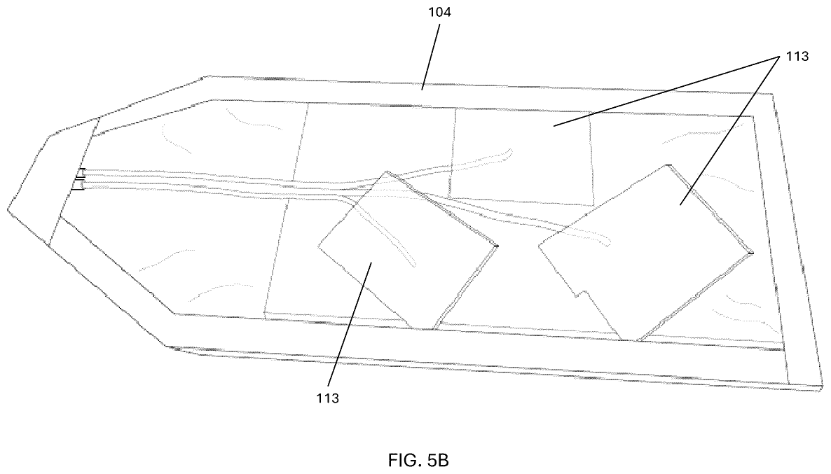

Sensing array 108 may include any number of sensors to detect an adhesion state of the sensing array 108 and more generally, the enclosure 104 to the garment 101 of the patient/user. In various embodiments, one such sensor modality may be one or more motion sensors 128. For example, each motion sensor 128 may be an inertial measurement unit (IMU). Further, each IMU may include or be communicatively coupled at least one of an accelerometer, gyroscope, or magnetometer each configured to detect the relative movement through space of sensing array 108. As shown in FIGS. 4A and 5A, motion sensors 128 may be disposed on sensing array 108. For example, and without limitation, sensing array 108 may include a motion sensor 128 disposed at each end, one proximate the anterior surface of the wearer and one proximate the posterior surface of the wearer of the sensing array 108 (within enclosure 104). In various embodiments, the motion sensors 128 may be disposed at the anterior and posterior areas of the sensing array 108 to record kinematic and dynamic information about the sensing array 108 and patient's activity while being worn. In various other embodiments, more than two motion sensors 128, such as three, four, five, ten or twenty IMUs may be utilized on the sensing array 108 in order to detect and record more motion data for analysis.

The plurality of motion sensors 128 may provide adequate data to the CPU (to be discussed below) to detect an adhesion state of the sensing array 108 to the garment 101. The adhesion state may be communicated to one or more of a computer, mobile device, tablet, medical intervention system or the user through audio, haptic or visual cues, and denote proper orientation of the sensing array 108 onto garment 101 for incontinence monitoring. For example, and without limitation, when the sensing array 108 (within enclosure 104) is placed onto a garment, there may be associated rotation and translation of the sensing array 108, as such as shown in FIG. 2. The plurality of motion sensors 128 may detect and record said translation and rotation, and provide that data to the CPU and as motion associated with the device (108) being attached to the garment 101. In some embodiments, the motion sensor 128 (IMU) data may be used to recommend care actions adjacent to incontinence, such as turning a patient on a regular basis to reduce the probability of pressure sore development.

In various embodiments, another sensing modality may be employed to detect or contribute to the detection of adhesion state of sensing array 108. For example, and without limitation, one or more other sensors, such as touch, proximity or electrical sensors may be deployed on sensing array or electrically connected to sensing array 108.

As shown in FIGS. 4A and 5A, one or more impedance sensing regions 117 may be disposed on sensing array 108. Each sensing region 117 may be disposed at opposite ends of the sensing array 108 and perpendicular to the centerline along the sagittal plane. That is to say, each sensing region 117 may be disposed at the anterior most and posterior most positions on the sensing array 108 such that when worn on the garment 101, the sensing regions 117 are disposed proximate the waistband at the front and back of the wearer.

Each sensing region 117 may be configured for capacitive sensing. Said sensing regions 117 may detect electrical interactions with human presence, proximity, or contact with a human. These regions may be formed from conductive traces (i.e., strips). In various embodiments, sensing regions 117 may be formed from copper, silver gold or alloys including same. These sensing regions 117 may be placed beyond the absorbent region 102 of the garment 101 for closer proximity to a patient's body when the enclosure is positioned on the garment 101 and worn. Therefore, sensing regions 117 may also serve as reference environmental sensors for incontinence detection by other impedance sensing regions 116, which will be described below. These impedance sensing regions 117 may be exposed regions for direct contact with interfacing surfaces. Alternatively, these regions may be covered by some substrate. Said substrate may reduce onset of corrosion. Said regions may also be coated to be protected from corrosion, moisture penetration, or other environmental damages.

The capacitive sensing regions 117 may register a high signal when in proximity with the human 201, owing to the large capacitance of the human body. If the sensing array 108 (or the enclosure 104 carrying said sensing array 108) were to be removed or to incidentally fall off patient's garment 101, then the capacitive sensing may read a lower signal. The motion sensors 128 (IMUs) data may identify a significant change in angular or linear, in position, velocity, or acceleration to confirm the low capacitive readings from the sensing regions 117 and indicate sensing array 108 has been removed from proximity to the human 201 (and therefore garment 101).

This combination of capacitive and motion sensing detected by the sensing regions 117 and motion sensors 128, respectively, offers a method for doctors, nurses, medical providers, caregivers or the patient themselves to know whether sensing array 108 is adhered or whether disrobing may have taken place, which may be a possible behavior for people living with dementia, Alzheimer's Disease or other neurodegenerative disorders, or for patients of advanced age. The adhesion state may be detected by the combination of motion sensors 128 and sensing regions 117 and communicated by sensing array 108 via one or more forms of feedback on the sensing array 108 or an external component, such as the computer or mobile device communicatively coupled thereto. For example, and without limitation, the feedback corresponding to adhesion state may include audio, visual, or haptic feedback, or one or more alerts, messages, or notifications made available on the digital interface to be interpreted by a user, such as any medical provider or the patient themselves. If the incontinence monitoring system 100 is to be relied upon to give incontinence alerts, then it may follow that the sensing array 108 falling off of the patient's garment 101, whilst not in the purview of a nurse or caregiver, could obstruct detection of incontinence events.

In various other embodiments, sensing array 108 may include one or more radiofrequency (RF) sensors configured to operate on the millimeter scale. The RF sensors may be configured to detect proximity or contact with the human (or other biological wearer). In various other embodiments, the RF sensor may be configured to detect muscle contraction or relaxation associated with incontinence events, both urine and/or fecal, and constipation, among other muscular operation. These RF sensors may be used in conjunction with or in place of impedance sensing regions 117 and the data captured therefrom may be similarly fed to CPU 132 or another processor to detect incontinence events and/or adhesion state of the system.

In various embodiments, incontinence monitoring system 100, and specifically sensing array 108, may comprise multiple sensors of different types to detect, alone or in combination, at least one of the presence, location, and amount of urine, feces or both, in garment 101. A multiplicity of sensors types, including multiple sensors configured to detect identical or similar phenomena, reduces the probability of false alarms or missed incontinence events, which could disrupt a doctor, nurse, medical provider, patient or caregiver's routine, as well as increase the probability of a patient contracting an avoidable complication, such as a urinary tract infection from prolonged exposure to soil.

In various embodiments, as will be described below, the arrangement of sensors of the sensing array 108 may correspond to human anatomy. In various embodiments, such as that shown in FIG. 4A, sensors and other electronic components may be arranged to be collinear with each other along a central axis lying the sagittal plane, parallel with said central axis, or symmetric about said central axis. This placement may reflect the urethra and the anus's general alignment with the human body's sagittal axis and may better position sensors with respect to the orifices from which waste exits the body. Said sensor layout may encompass the overall length of the absorbent region of a garment, which may increase the sensing array 108's ability to detect urine, feces or both. In various embodiments, decoupling capacitors may be connected to electronic components to stabilize power signals, wherein said capacitors may be placed near electronic components for better performance and augment the sensing array 108's reliability. The use of a multiplicity of sensors may also augment the ability of the sensing array 108 to be used across different brands of incontinence garments by reducing dependence on a singular sensing method.

In various embodiments, the multiplicity of sensor types to detect incontinence events may include temperature, capacitive, and gas sensing methods. Alternative embodiments may also utilize electrostatic charge sensing as a supplement to the above.

As shown in FIG. 4A, sensing array 108 may include a plurality of temperature sensors 112. The plurality of temperature sensors 112 may be arranged along the centerline of sensing array 108 from the anterior end to the posterior end thereof. Each temperature sensor 112 of the plurality of temperature sensor 112 may be arranged regularly between the front end and the back end of the sensing array 108. For example, and without limitation, the plurality of temperature sensors 112 may be positioned such that the anterior most temperature sensor 112 is located close to the area of the garment 101 closest to the urethra and the posterior most temperature sensor 112 is located proximate the garment 101 closest to the anus of the user. In various embodiments, the plurality of temperature sensors 112 may be clustered proximate the front or the back of sensing array 108. In various other embodiments, a plurality of clusters or segments of linearly arranged temperature sensors 112 may be arranged on the sensing array 108. Due to the encapsulation of the sensing array 108 within the enclosure 104 which curves along the body of the patient, the plurality of temperature sensors 112 may be similarly curved in space such that they are retained in close proximity to the garment 101 when enclosure 104 is adhered to garment 101.

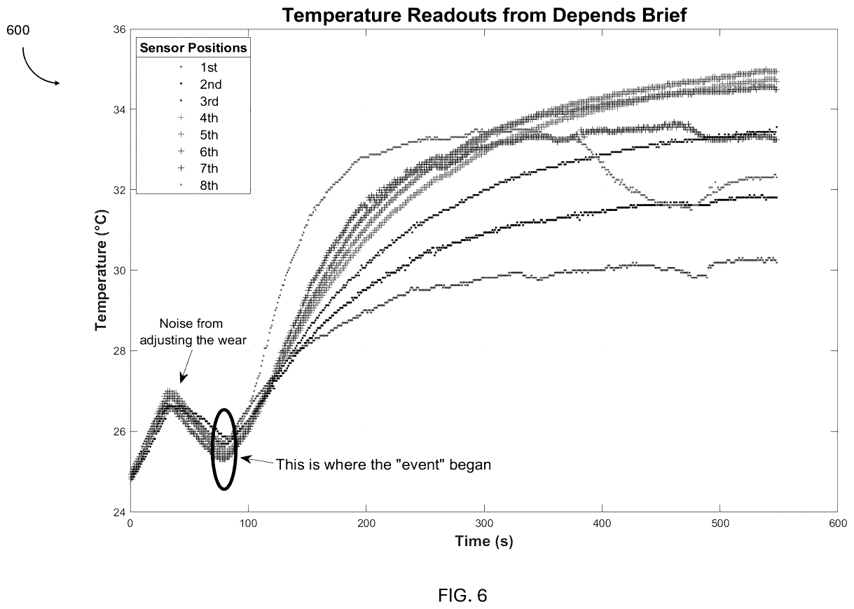

In various embodiments, each of the plurality of temperature sensors 112 may detect thermal energy through conduction, a plot 600 of exemplary temperature readings from a plurality of temperature sensors 112 is shown in FIG. 6. In various other embodiments, at least one temperature sensor 112 of the plurality may be an infrared (IR) sensor configured to detect temperature. In those implementations, the IR temperature sensors 112 may be disposed elsewhere on garment 101, enclosure 104 or sensing array 108 and oriented towards the areas of interest associated with incontinence events. The plurality of temperature sensors 112 may be arranged in a collinear fashion with respect to each other to identify a change in the state of a garment 101 by the introduction of urine and/or feces as a heat source, which may increase the temperature of the absorbent region 102 of the garment 101 in the areas insulted by soil. In various embodiments embodiment, temperature sensors 112 may also be placed in line with a CPU and aligned with the centerline of garment 101 as shown in FIGS. 4A-4B, when the sensing array 108 is placed within enclosure 104. A temperature change may be detected by transient heat transfer via conduction, as urine and/or feces may be captured by the absorbent region 102 of the garment 101 and remain so until said garment 101 is changed.

In various other embodiments, one or more temperature sensors 112 may be disposed out of line with centerline of sensing array 108, such as the arrangement shown in FIG. 4B, where two temperature sensors 112 are positioned laterally from the centerline to detect thermal signatures away from the sagittal plane.

Fourier's Law of Conduction may be used to justify temperature sensing with the introduction of waste as a heat source in the garment. Assuming no internal heat generation, thermal mass of soil may be related to transient conduction through the garment's absorbent region 102 to the garment 101's exterior surface temperature, which may yield an exponential relationship between the temperature at the exterior of the garment 101 and the introduction of urine and/or feces therein.

Temperature may be employed as a favorable method of detection because urine and/or feces both exit the body at core temperature, which is usually higher than skin temperature, unless a person is exposed to an external heat source. Both forms of excreta (urine, feces) are water-based compounds—urine is reported to be 95-98% water-based, while feces may vary in water content, generally about 70% on average in some studies. The thermal properties of waste imply that urination or defecation introduce a higher temperature signal to a garment than was present while said garment was clean. Given the specific heat capacity of water being approximately 4.184 J/gK, urine and feces both hold their temperatures for extended durations of time compared to substances with lower specific heat capacities. This thermal property may permit differentiation of urine and/or feces from other substances by comparing characteristic response times. The high specific heat capacity suggests that temperature signals for urination or defecation may remain in a high state for an extended period of time.

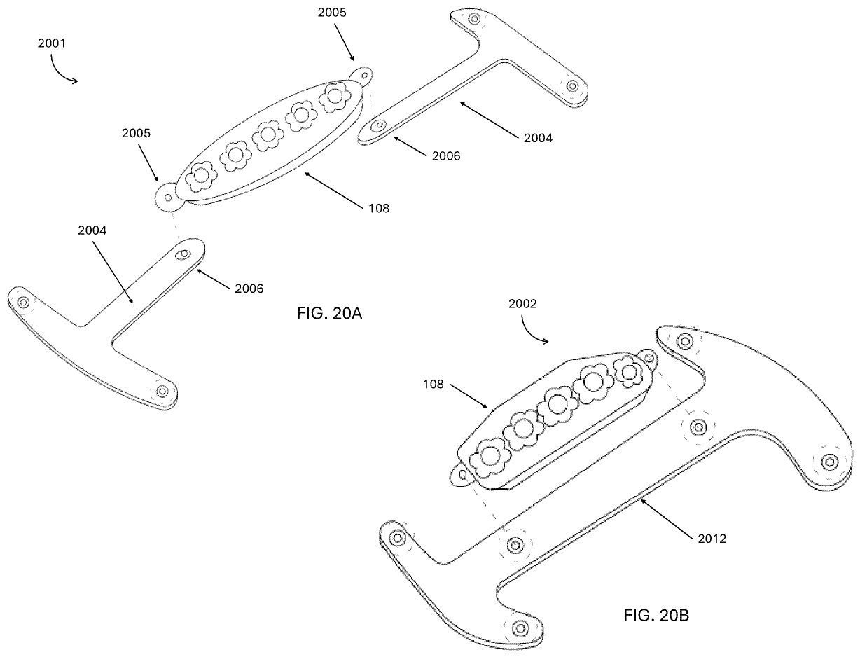

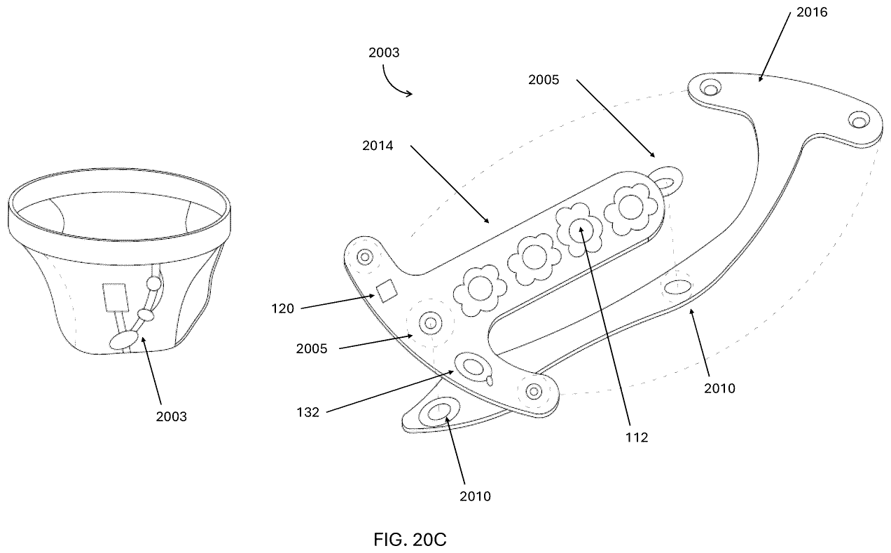

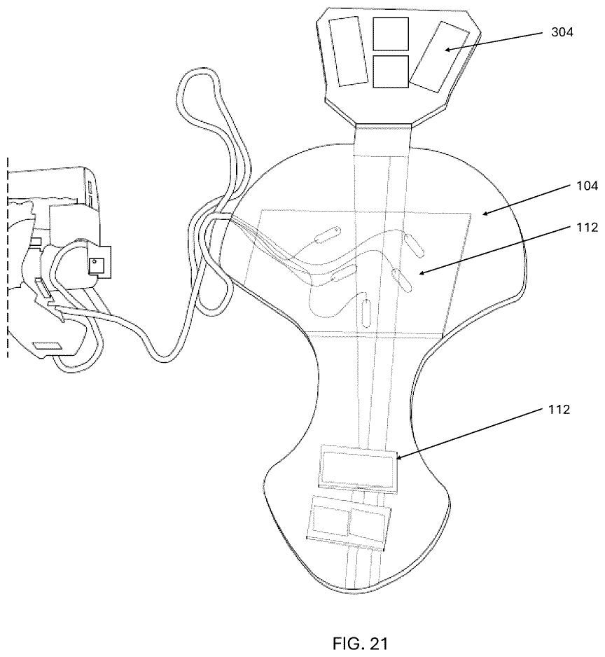

Further, since the temperature readings may depend on the density of the heat source's material, the temperature readings may be independent of the volume of soil introduced and may instead be dependent on the placement of temperature sensors relative to the location of the soil. Since the plurality of temperature sensors 112 may be employed as shown in FIGS. 4A-4B (and FIGS. 5A, 20A-20C) the presence of urine and/or feces may be identified via an increased temperature signal relative to baseline and that mimics the characteristic exponential curvature associated with transient conduction as tabulated above.

Further, the relative location of soil may be established by comparing readings between adjacent temperature sensors 112 of the plurality of temperature sensors 112. For example, and without limitation, if a given temperature sensor 112 node registers an exponential profile, but an adjacent temperature sensor 112 does not register a similar profile, then that difference may speak to the relative location of soil based on the physical location of the sensors in question. In a further example, if an external reference point is taken, such as the CPU, then the location of soil within the garment 101 relative to the absorbent region 102's perimeter may be estimated with temperature sensing via the plurality of collinearly arranged temperature sensors 112. Thus, one or more of the temperature sensors 112 nearer to a patient's anterior surface (front of user) may identify urine, whereas one or more of the temperature sensors 112 nearer to a patient's posterior surface (rear of user) may identify feces.

In order to account for variance across garment manufacturers, which may utilize different materials with specific and proprietary thermal and material properties, each of the plurality of temperature sensors 112 may be positioned optimally for sensing one or both of urine and feces. Further, one or more processors in communication with the plurality of temperature sensors 112 may appropriately boost or suppress signals based on relative positioning of each of the sensors along the centerline of the sensing array 108.

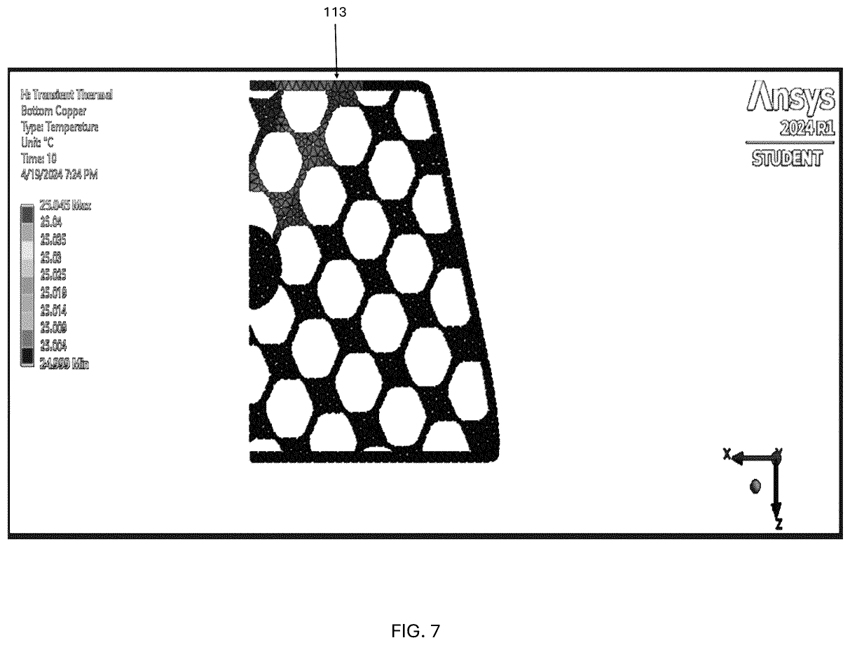

In various embodiments, sensing array 108 may include an overlay element 113 may be positioned over at least a portion of the plurality of temperature sensors 112. The overlay element 113 may be formed as a lattice, mesh, or otherwise conductive, planar component that touches or is near to, at least a portion of the plurality of temperature sensors 112. In various embodiments, overlay element 113 may include a geometrical patterned body made of a material with high thermal conductivity, such as copper, silver, gold or a combination thereof, among others. Said overlay element 113, may be placed over temperature sensors 112 to expand the area of detection and facilitate heat transfer directly or near to the temperature sensors. A schematic representation of sensing array 108 is provided for clarity, which shows an exemplary overlay element 113 covering the plurality of discrete nodal temperature sensors 112. Image 700 of FIG. 7 depicts thermal simulation data showing in-plane heat transfer through the geometrical pattern of overlay element 113 that may designate nodes, wherein said nodes could correspond to temperature sensing elements. In some embodiments, said overlay element 113 may be continuous and/or connected to multiple temperature sensors. In various other embodiments, said overlay element may be a series of discrete, physically isolated (non-connected) shapes placed above individual temperature sensors, such as the embodiment shown in FIG. 5B. In various embodiments, said overlay element 113 may employ radial symmetry. In some embodiments, said overlay element 113 may employ a gradual increase in width or cross-sectional area from the extremities, perimeter, or fringes towards the center of said geometry, and said center may be positioned over a given temperature sensor 112. In various embodiments, said overlay element 113 may include biomimetic designs. In various embodiments, said overlay element 113 may resemble flora, leaves, vegetation or flowers with a certain number of petals. In some embodiments, said overlay element 113 may be hollowed out in certain regions to drive heat transfer towards a given temperature sensor 112.

The overlay element 113 may be used to conduct heat from one area of the sensing array 108 towards at least a portion of the temperature sensors 112 on which it is overlaid. This overlay element 113 may aid the plurality of temperature sensors 112 in detecting a thermal signature of a remote area in the garment 101 which may otherwise go undetected due to the limited physical area and discrete location of the temperature sensors 112. Additionally, overlay element 113 may aid in conducting thermal energy towards the temperature sensors that may otherwise go undetected due to the inefficiency of in-plane heat transfer within absorbent garments, which are typically composed of relatively low thermal conductivity fabrics.

With continued reference to FIGS. 4A and 5A-5B, sensing array 108 may include impedance sensing regions 116, 117 and 118 positioned thereon. Impedance sensing regions 116, 117, 118 may be configured for capacitive sensing (and thus interchangeably referred to as capacitive sensing regions) of one or more of incontinence events and/or adhesion state of the system 100. As shown in FIG. 4A and 5A-5B, sensing array 108 may include at least two capacitive sensing regions 116a and 116b. Each capacitive sensing region 116a, 116b may be collinear with one another and embodied as conductive traces. Said capacitive sensing regions 116a, 116b may be arranged parallel to the centerline of the sensing array and therefore parallel to the sagittal plane. Sensing region 116a may be disposed proximate the front of sensing array 108 for detection of urinary incontinence events (i.e., the presence of urine) and sensing region 116b may be disposed proximate the back of sensing array 108 for detection of fecal incontinence events (i.e., the presence of feces).

In various embodiments, a plurality of capacitive sensing regions 116 may be employed for either form of excreta, or for both, which may promote forms of sensing with amplified signal-to-noise ratios, such as differential sensing. Physical clearance may be specified between regions to ensure each signal is discrete. In some embodiments, resistors or inductors may be included to alter the frequency of capacitive sensing. In some embodiments, an external oscillator or pulse width modulator may be included to alter the frequency of capacitive sensing, which may reveal resonant frequencies associated with incontinence event procession.

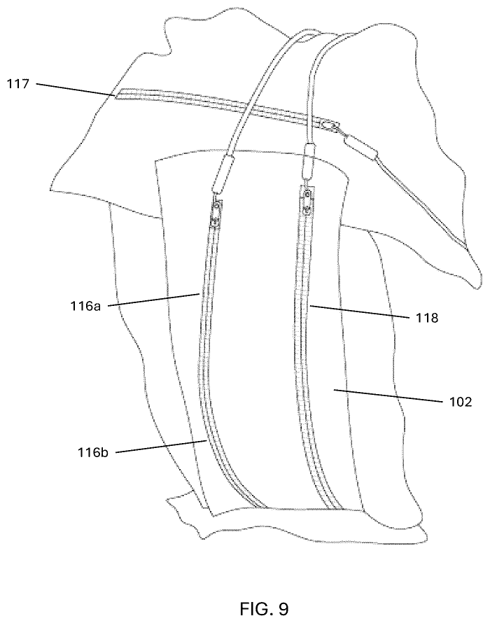

In various embodiments, capacitive sensing regions may be embodied strips of conductive materials. In some embodiments, said trace or strip lengths may not exceed half of the overall length of the absorbent region 102 of a garment 101 to reduce the chances of misregistration of a urinary or fecal event for the other. In various embodiments, the conductive traces may be made from copper or another conductive material, or composite material, and may serve as data channels. In various embodiments, said traces may be interpreted as analog signals or as digital signals. Analog signals may be translated to digital signals with a capacitance-to-digital converter. In various embodiments, said traces may be sized and shaped with a rectangular planform to permit simple calculation and estimation of capacitive readings. In various other embodiments, said trace may also be cut with a non-rectangular shape. In various embodiments, sensing regions 116a, 116b may be positioned collinear with one another and collectively parallel a common ground bus 118 or separate ground traces. A simplified representation of the parallel arrangement between sensing region 116a, 116b and ground trace 118 is shown in FIG. 9. In some embodiments, the geometry of the data channels or the ground bus 118 may differ from the present embodiment in length, width, characteristic shape, or distance between electrodes. In some embodiments, traces may be arranged collinear with said central axis of the sensing array 108, and cutouts may be made within said traces to avoid physical or electrical interference with other components on the sensing array 108.

This co-planar arrangement of copper strips for data channels and for a ground bus 118 may form a capacitor, where the electric field between the data channels 116a, 116b and the ground bus 118 may follow a radial arc pathway due to fringe field effects. The electric field lines may pass through each of the garment 101's layers, including through the absorbent region 102, as well as through a person's body when the sensing array 108 is worn. The sum of the dielectric constants of each material through which the electric field lines pass may inform the overall capacitance readings. This means that capacitive sensing may identify the presence or the absence of a given material.

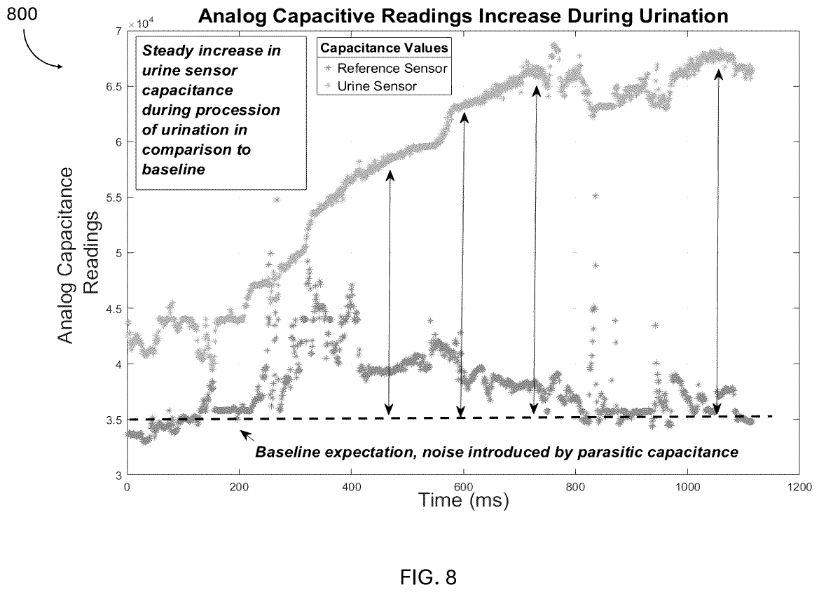

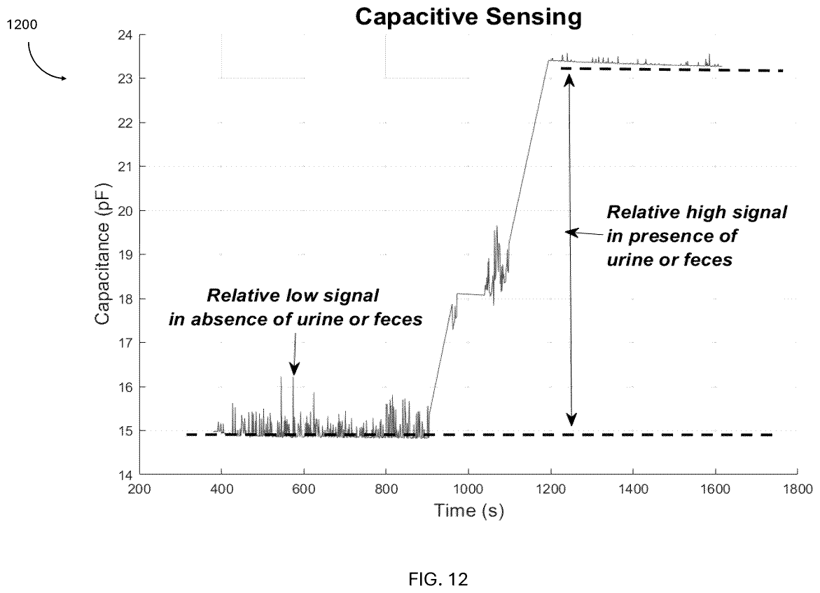

Capacitance may increase when electric field lines pass through materials with higher dielectric constants, which are inherent material properties. This is because materials with higher dielectric constants may serve to route electric field lines more strongly from a data electrode 116a, 116b to a ground electrode 118, thereby increasing the strength of fringe field effects. Air has a characteristic dielectric constant of one, with many fabrics hovering around 1.1 as their dielectric constants. Water has a characteristic dielectric constant near 80 at standard conditions for temperature and pressure, which represents an 80-fold multiple atop the dielectric of air within a clean garment. Since urine and feces are both water-based, particularly urine, both forms of excreta may yield high, discernable capacitive signals when introduced to a previously clean garment, as shown for example, in plot 800 of FIG. 8 and plot 1200 of FIG. 12.

Since multiple electrodes may be used, with an intentional gap optionally left between said electrodes to avoid physical contact, and since each electrode is designated using a separate channel for signal output, the relative location of soil within a garment can be established to help determine whether a patient had a urinary, fecal, or dual incontinence event, which informs different care needs. An electrode (sensing region 116a) closer to the front of a patient's body that registers a high signal may be interpreted as a urinary event, whereas an electrode (sensing region 116b) near said patient's rear may denote a fecal event.

Capacitance may also exhibit minimal lag time compared to temperature sensing, which may serve as an excellent starting point for detecting an incontinence event with swift response. Thus, the combination of capacitance and temperature may be used to establish the presence and location of soil to distinguish urinary and fecal incontinence events.

Unlike temperature, which is agnostic to the volume of soil present, capacitance is volume dependent. As the volume of soil present in a garment 101 increases, so too may the capacitance reading. In a sense, the capacitance may step to a higher signal reading and may hold said reading for as long as a given volume of a substance is present.

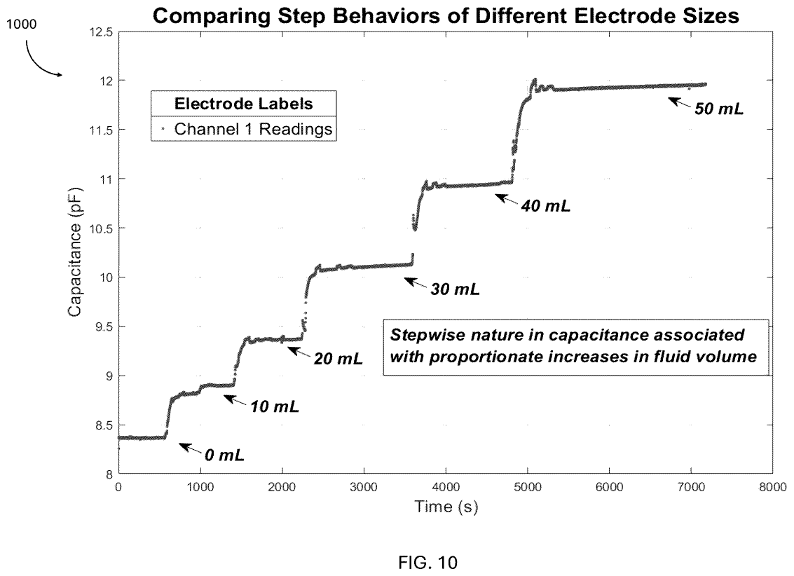

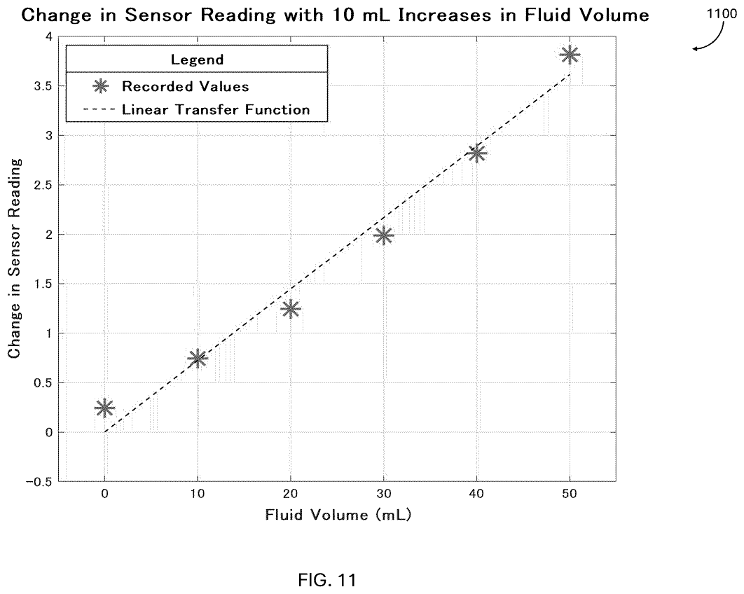

From this stepwise behavior, shown in plot 1000 of FIG. 10, a linear correlation between capacitance and fluid volume outputs can be established. This is aided by the summation of the dielectric constants of material through which the capacitor's electric field lines pass. Given a system with multiple materials of varying dielectric constants, the increasing presence of a specific material with a known dielectric constant may increase the capacitance proportionally. Linearity for a coplanar capacitive sensing arrangement can be expressed with linear regression. Linear regression may then be used to estimate the volume of soil in a garment. Integration may also be used to estimate the volume of soil in a garment. The graph 1100 of FIG. 11 depicts a linear trend of increasing capacitance for 10 mL increases of water, which closely approximates the dielectric constant of urine.

Contact pressure between a substance being measured and a data electrode may increase capacitance read by the sensor. Since soil may increase the weight of garment 101 when captured, contact pressure of said garment 101 with capacitive sensing (such as regions 116a, 116b) may also stand to increase as long as the sensing array 108 remains adhered to the garment 101 via enclosure 104, thereby increasing capacitance values. Thus, capacitance may present a marker that can tell the presence, location, and amount of urine and/or feces within a garment without direct contact.

In various embodiments, filtering and/or shielding may be employed in order to reduce or remove the parasitic capacitance due to the proximity of the human user to the sensing regions 116a, 116b. While excreta, such as urine and/or feces, may still be detected, additional filtering may be needed to remove the effects of parasitic capacitance owing to human proximity. Further, shielding may be needed to reduce the effects of said parasitic capacitance, both from humans and from other circuit board elements. In some embodiments, shielding may be achieved with active shield drivers. In some embodiments, shielding may be achieved with grounded conductive material.

Since both urine and feces are water-based, it may be possible that incorrect orientation of the sensing array 108 within enclosure 104 onto garment 101 could cause urination to be registered as defecation, though there is enough variance in the water content between urine (95-98% water content) and typical feces (70% water content) to register distinct signals. On the subject of incorrect orientation, diarrhea may register as being similar to urine if solely dependent on temperature and capacitance. Therefore, increased confidence in incontinence detection may be derived by implementing one or more further sensor modalities that can distinguish urinary and fecal characteristics.

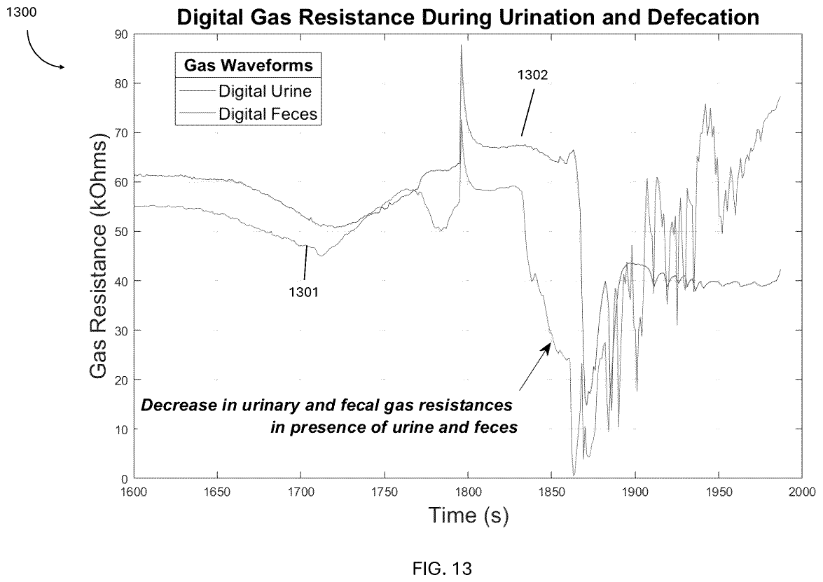

With continued reference to FIGS. 4 and 5A, sensing array 108 may include one or more chemical sensors. For example, and without limitation, sensing array 108 may include at least one gas sensor. In various embodiments, one or more digital gas sensors 120 may be disposed within sensing array 108 configured to detect gaseous compounds expelled in associate with urination and/or defection, as shown in plot 1300 of FIG. 13, where feces trace 1301 and urine trace 1302 are shown decreasing in gas resistance in the presence of urine and feces. In various embodiments, one or more analog gas sensors 121, 131 may be disposed on sensing array 108. In various embodiments, one or more of each of digital gas sensors 120 and analog gas sensors 121, 131 may be employed within system 100.

Both urine and feces have metabolomes encompassing volatile organic compounds (VOCs), which affect air quality readings picked up by said digital sensors. Some air quality sensors may represent signals as a resistance which decreases with the presence of VOCs, while other air quality sensors may represent signals as a conductance which increases with the presence of VOCs. In any case, the main implication may be a discernible change in air quality sensor reading when air is fouled by VOCs from urine or feces. A method of chemical sensing may be included to identify urination or defecation. In various embodiments, system 100, specifically sensing array 108, may include two digital gas sensors 120 configured to detect air quality or be air quality gas detectors. As shown in FIGS. 4A and 5A, a first digital gas sensor 120 may be placed at the front end of the sensing array 108, and a second digital gas sensor 120 may be placed at the rear end of the sensing array 108. In various other embodiments, more than one digital gas sensor 120 may be placed at either or each end of the sensing array 108.

In various embodiments, each digital gas sensor 120 may be disposed on sensing array 108 such that it is located proximate the waistband of the garment 101 for airflow or comparative considerations with the ambient gaseous environment.

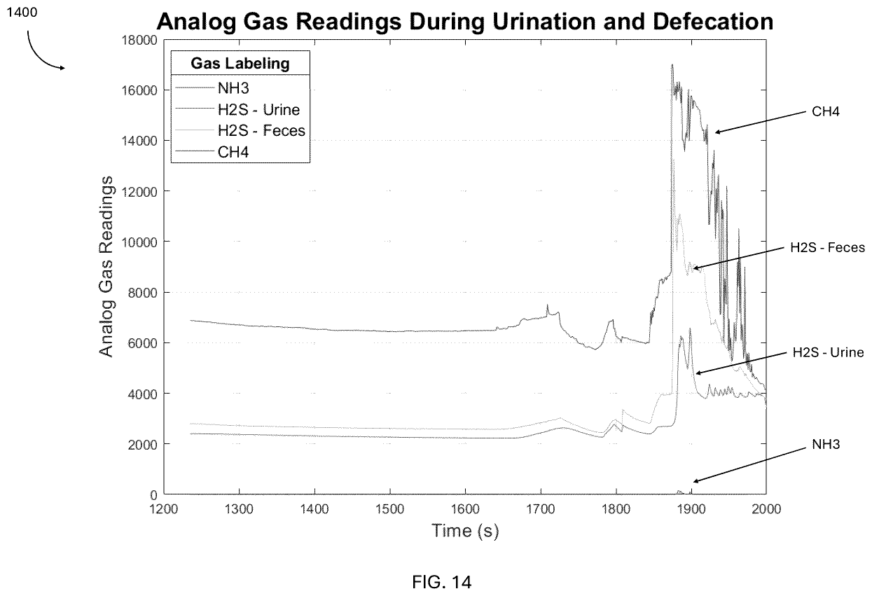

Sensing array 108 may further include one or more analog gas sensors 121, 131 configured to detect gases associated with urination and/or defecation, as shown in plot 1400 of FIG. 14. In various embodiments, additional analog gas sensors 121, 131 may also be employed in conjunction with the digital gas sensors 120. For example, and without limitation, at least one analog gas sensor 121 may be positioned towards the front of the sensing array 108. In various embodiments, each gas sensor, such as digital gas sensors 120 or analog gas sensors 121, 131, may be configured to detect a predetermined or expected gaseous compound associated with excreta or expected location of excreta. For example, one or more gas sensors configured to detect ammonia (NH3) may be employed for urine and placed proximate the sensing array 108 that overlays the anterior surface of the user (close to the urethra). In various embodiments, one or more gas sensors may be positioned proximate the anterior surface of the user on sensing array 108 that is configured to detect a dihydrogen sulfide (H2S) associated with urine. In various embodiments, one or more gas sensors may be proximate the anterior surface of the user on sensing array 108 configured to detect an odor or volatile organic compounds (VOC) associated with urine. In various embodiments, one or more gas sensors may be proximate the posterior surface of the user on sensing array 108 configured to detect an odor or volatile organic compounds (VOC) associated with feces. In various embodiments, one or more gas sensors may be proximate the posterior surface of the user on sensing array 108 configured to detect methane (CH4) associated with feces. In various embodiments, one or more gas sensors may be proximate the posterior surface of the user on sensing array 108 configured to detect H2S associated with feces. Each of the gas sensors disclosed herein may be used in isolation or in any combination.

In various embodiments, analog sensors may be connected to analog-to-digital converter (ADC) modules, which may increase the resolution of analog signal readings to augment gas signal quality.

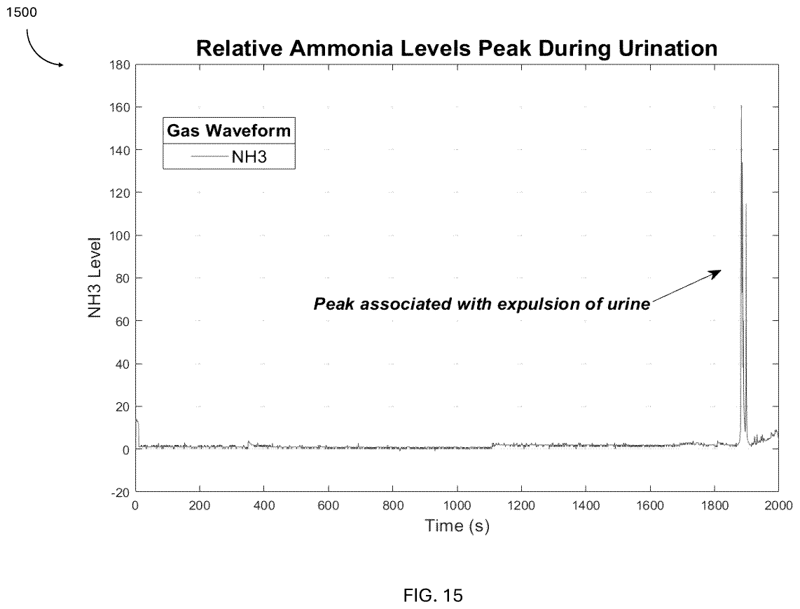

During urination and defecation, different gasses may build up as processing of waste in each bodily system occurs. Urine constitutes the primary source of nitrogenous waste in the human body, with various studies estimating that nitrogen comprises 14-18% of dry solids. Leading nitrogenous compounds in urine include urea, ammonia, and creatinine. Urea and ammonia may both form by way of the breakdown of amino acids in the kidneys, though urea may be in the form of dissolved solute in urine, while ammonia may be generated in gaseous form in the kidneys and can present in gaseous form with expulsion of urine from the urethra, as shown in plot 1500 of FIG. 15. Further, once urine is expelled from the body, if urine is left standing, then urease enzymes can further lyse urea, which may form ammonia as a byproduct. Urine may also contain sulfur, which can be present in dissolved sulphates or as H2S produced in the kidneys and then expelled with urination.

Feces' dry matter is predominantly composed of carbonic compounds, with some studies estimating 40-55% carbon composition of dry solids. Methane is one such compound, which has been documented in studies concerning biofuel applications of feces. Methane is not only produced as part of fecal solids, but methane is also produced by gut bacteria in the gastrointestinal tract and expelled in gaseous form with contraction of the sphincter to eject stool, which justifies the implementation of a methane sensor for fecal incontinence monitoring.

Gut bacteria play a significant role in differentiating urinary gas sensing from fecal gas sensing. Some studies report that gut bacterial products may contribute to 92% of the fecal metabolome, compared to just 3% of the urinary metabolome. The fecal metabolome may also be characterized by high presence of volatile organic compounds (VOCs) that contribute to feces' characteristic odor, hence why the present embodiment may include a sensor capable of reading odor or VOC presence.

Sulfuric compounds may be one of the contributing portions of the fecal VOC profile, including H2S, which is produced by gut bacteria in the gastrointestinal tract during the catalyzing of sulfur-containing amino acids and may therefore be another viable candidate for detecting fecal incontinence.

The characteristic spikes of CH4, H2S for feces, H2S for urine, and NH3 may speak to an increase in gas sensor reading according to urination or defecation taking place, with the expulsion of respective gasses. In some cases, H2S may be double-registered, depending on the ability of gas to diffuse from either urine or feces to both H2S sensors. Thus, deployment of CH4 and NH3 gas sensors in tandem may help to address potential false alarms.

The placement of gas sensors may be collinear with the centerline of the sensing array 108 or parallel to said centerline. In various embodiments, either digital gas sensors 120 or analog gas sensors 121, 131 may be interposed with the plurality of temperature sensors 112. In various embodiments, the placement of gas sensors (120 or 121, 131) may extend beyond the perimeter of the absorbent region 102 of the garment 101. For example, and without limitation, gas sensors 120, 121, 131 may be located on the brim of the garment 101 proximate the waistline, as this may provide more advantageous airflow from the interior of garment 101 to the exterior of said garment 101. This may be especially useful because some garment manufacturers may advertise forms of “odor control,” wherein said odor from excreta may be trapped within the absorbent region 102 of a garment 101. This may make it difficult to justify placing gas sensors within the perimeter of the absorbent region 102 of a garment 101 because diffusion of odor may be intentionally impaired within said region. Therefore, in various embodiments, gas sensors may be placed along other portions of sensing array 108 proximate one or more of the brim of the garment 101 and along the sides of a person 201's buttocks, which may permit more airflow exiting said garment. An example enclosure 104 shaped to accommodate this relatively wider arrangement of sensors is shown, for example, in FIG. 21. In various embodiments, extensions onto the sensing array 108's substrate and electrical connections, in a direction that is perpendicular or non-parallel to said array's central axis may be included for adequate placement of gas sensors.

Further, gas sensors including vents for ingestion or exposure to the gaseous compounds to be detected may be employed in sensing array 108. In various embodiments, any gas sensor or the plurality of gas sensors may be oriented such that their vents face into the garment 101's exterior surface, which may offer a direct path for gas diffusion to a respective sensor. Alternatively, one or more of the gas sensors may be placed such that their vents face away from the garment 101's exterior surface to read the state of the air surrounding the person 201's garment 101, thereby depending on diffusion of gasses out of said garment. In various embodiments, a gas membrane, or a material that is permeable to air and gases, may be placed over the vents of at least one of the gas sensors to protect said sensors from collecting dust, moisture, or contamination in their openings.

Gas sensors may exhibit an Arrhenius relationship, wherein the sensor readings vary with respect to time based on the concentration of a certain gas present. Said relationship may appear as an exponential relationship.

In various embodiments, sensing array 108 may include a region designated for electronics housing. As shown in FIGS. 4A and 5A-5B, said designated area may be at the anterior-most or posterior most ends of sensing array 108 such that the electronics housing is positioned proximate the waistband of a wearer when enclosure 104 is attached to garment 101. In various embodiments, the electronics housing may be placed on the end of the sensing array 108 that is intended to be worn against the front of a patient's garment 101, located away from the absorbent region of a garment or around a patient's bladder. This placement may reduce the risk of pressure sores that could be incurred from a patient sitting or lying in place, on their sides or on their back for extended periods of time. In various embodiments, sensing array 108 may include a central processing unit (CPU) 132, a battery 140 and additional peripherals to be described below.

In various embodiments, CPU 132 may be capable of reading sensor data as detected by the one or more sensors described above, performing mathematical operations, and boasting a wireless communication module 136 (such as an antenna) to send alerts of changes in device or patient status to one or more user devices, such as a computer, mobile phone, tablet or the like. In some embodiments, one or more thermal management components may be employed to reduce the temperature of the CPU 132, such as a physical external heat sink or, if available, connection of the CPU 132's thermal pad to a part of the sensing array 108, such as a ground plane, or to some heat sink. Said method of thermal management may confer additional risk mitigation in limiting the chances of a patient experiencing burns while wearing said sensor array 108, which is a significant risk for other enuresis alarms, or bedwetting monitors, per the FDA's medical device failure report database. The placement of the CPU 132 into enclosure 104, (which may be embodied as a fabric sleeve) may help to further reduce burn risk, as fabric materials typically confer appreciable degrees of thermal insulation and may thus inhibit heat transfer from the CPU 132 in the direction of a patient's skin. A CPU temperature sensor 164 may be connected to the CPU 132 to monitor its temperature. Said component may take the form of a thermistor or a temperature sensor. Said CPU temperature sensor 164 may issue an alert or an interrupt upon CPU 132 temperature exceeding a predetermined or dynamic threshold temperature.

In some embodiments, the CPU 132 may have a built-in or onboard antenna 136 for wireless communication. In some embodiments, an external antenna or a wireless communication module 136 may be electrically coupled to the CPU 132 to permit wireless communication. In some embodiments, there may be a designated region not containing conductive material to avoid interference with said antenna 136. In some embodiments, a passive radio frequency system may be employed to communicate wireless information, wherein said system may be charged to transmit upon detection of an incontinence event. In some embodiments, the CPU 132 may be oriented such that its topmost face or any substantially planar portion thereof faces into garment 101's exterior, to affect a sleek form factor and increased comfort when worn. In some embodiments, the CPU 132 may be oriented such that its topmost face or the substantially planar portion thereof faces away from the garment 101's exterior, to increase resistance to heat transfer between the CPU 132 and the garment 101 to reduce burn risk.

In various embodiments, battery 140 may use a lithium-based battery chemistry. In various other embodiments, a non-lithium-based battery chemistry may be employed. In various embodiments, the battery 140 may be removable to for charging and/or replacement. In various embodiments, the battery 140 may be rechargeable or fixed in place within the electronics housing or a separate compartment of sensing array 108 or enclosure 104. In various embodiments, the battery 140 may have the form factor of a coin cell or a button battery. In various embodiments, the battery 140 may be placed in line with the centerline of the sensing array 108. In various other embodiments, the battery 140 may be disposed alongside the centerline of the sensing array 108. In some embodiments, a connector, two conductive leads or strips of conductive material, may be placed on the sensing array 108 and made accessible to facilitate connection of an external battery 140 or a battery pack to said sensing array 108.

Sensing array 108 may include other peripherals electrically connected thereto and carried thereon. The peripherals may be disposed a sufficient distance away from any sensors designated for incontinence detection, such as temperature, capacitive or gaseous sensors as described. In various embodiments, sensing array 108 or another portion of system 100 may include one or more components for hardware authentication to enable use and ensure cybersecurity of the patient and the related computing systems in communication with the system 100.

For example, and without limitation, sensing array 108 may include one or more of a real time clock, timer, or similar time-keeping electronic component (collectively, timing element 148) to timestamp device firmware activities and sensor readings. In various embodiments, sensing array 108 may contain a designated memory storage module 160 to expand the memory storage capacity available on the sensing array 108. In various embodiments, said memory storage module 160 may include or receive an external memory device, such as an SD card module to record sensor data and device firmware actions. In various embodiments, sensing array 108 may include a battery management component, such as a subcircuit, to estimate or monitor the battery capacity remaining. Said component could be a battery fuel gauge to inform nurses, doctors, medical providers, the patient themselves, and/or caregivers of the device's battery level so that the battery may either be recharged or replaced. Alternatively, a subcircuit may be included to measure current draw from or voltage difference across a battery cell or multiple cells. This embodiment may contain a multiplexer 144 to resolve address conflicts between repeated sensors. Said multiplexer 144 may also be used to reduce the current draw of the sensing array 108 by polling a portion of the sensors on the sensing array 108, rather than polling every sensor on said sensing array 108. Said reduction in current draw may be favorable to reduce heat dissipation from the sensing array 108 and therefore reduce some risk of burns to a patient's skin.

For both incontinence event detection and adhesion state detection, thresholds may be specified by one or more medical providers or automatically by sensing array 108 for each sensing modality to determine cut-offs between traits of a clean garment 101 and traits of a soiled garment 101. For example, and without limitation, sensor interrupts may be employed to send alerts to the CPU 132 upon a sensor reading exceeding, meeting, or falling below a given threshold for that sensor modality. In various embodiments, each sensor may be continuously polled in a loop for comparison with a given threshold or result of a mathematical process. In various other embodiments, a subset of the sensors may be polled continuously, and other sensors may be polled discretely. For example, and without limitation, the plurality of temperature sensors 112 may be polled continuously and the various impeding sensing regions 116, 117 may be polled discretely. In another example, the opposite polling methodology may be carried out.

In various embodiments, detrending or linearization may be employed on sensor data to account for sensor drift. In various embodiments, averaging may also be employed on sensor data. In various embodiments, frequency domain analysis or fast Fourier transforms may be performed on sensor data. In various embodiments, exponential regression may be employed on temperature sensing or gas sensing, for example. In various embodiments, linear regression could be used for temperature sensing or gas sensing after taking the logarithm of each respective dataset. In various embodiments, differential sensing may be employed on capacitive sensing, e.g., on the sensing regions 116, 117. In various embodiments, derivatives or integrals of sensor data may be approximated to identify changes in the state of each sensor profile in order to detect incontinence events and/or adhesion state. For example, IMU linear and/or rotational velocity and/or acceleration may be integrated to estimate the change in position of sensing array 108 while being worn.

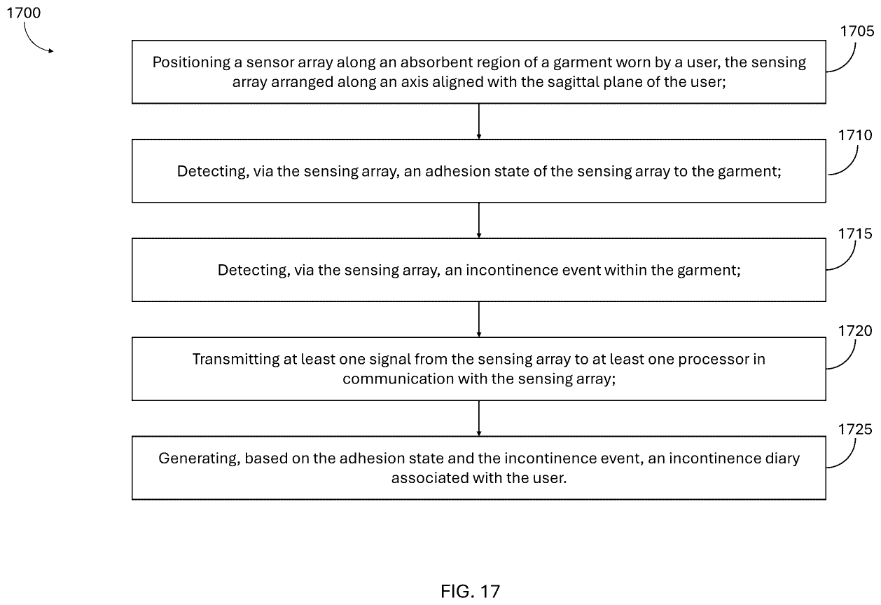

Referring now to FIG. 17, a flowchart depicting an exemplary method of incontinence monitoring is shown. Method 1700 may include, at step 1705, positioning a sensing array along an absorbent region of a garment, the sensing array arranged substantially along an axis aligned with a sagittal plane of a user wearing the garment.

In some embodiments, the incontinence sensing array 108 may be adhered to the garment 101 prior to said garment 101 being dressed onto a patient. In some embodiments, the sensing array 108 may be adhered to the garment 101 while a patient is already wearing said garment. Once the sensing array 108 is attached onto a patient's garment 101, a notification may be given on the user interface confirming that sensing array 108 is adhered to a given patient. If device adhesion to a garment being worn by a patient is determined to be lost, which may be determined by firmware on the CPU, the CPU may send an alert to update the user interface with a status alluding to a device losing contact with a patient's body.

The sensing array may be sensing array 108 as described herein. In various embodiments, the sensing array 108 may include a first end proximate an anterior surface of the user and a second end proximate a posterior surface of the user. As discussed above, the sensing array 108 may be encapsulated within an enclosure 104, such as a flexible fabric sleeve.

Accordingly, in various embodiments, sensing array 108 may be embodied as a PCB, a flexible PCB, or a series or rigid components flexibly coupled to one another to conform to the anatomical features of a user proximate to the orifices in which excreta is expelled, e.g., the groin/perineum, buttocks, such that the sensing array is disposed near to the urethra and anus of the user. Further, as described above, the sensing array 108 may include a plurality of temperature sensors 112 arranged coaxially with the axis within the sagittal plane between the first end and the second end of the sensing array. In other words, the plurality of temperature sensors 112 may be aligned along the centerline of the sensing array 108. The anterior most temperature sensor may be positioned proximate the urethra of the user (over the garment 101 that overlays the urethra) and the posterior most temperature sensor may be positioned proximate the portion of garment 101 that overlays the anus of the user.

Further, as described above, sensing array 108 may include at least one impedance sensing region configured for capacitive sensing. In various embodiments, there may be a plurality of impedance sensing regions 116a and 116b aligned parallel to the centerline of the sensing array, a first of which nearer to the anterior surface of the user and configured to detect urine, and a second of which is positioned near the posterior surface of the user and configured to detect feces. In various embodiments, there may be a ground trace 118 aligned parallel with the impedance sensing regions 116a, 116b across the centerline of the sensing array 108. Further, there may be two or more impedance sensing regions 117 arranged perpendicular to the centerline of the sensing array 108. A first of these sensing regions 117 may be arranged at an anterior end of the sensing array 108 and a second of these sensing regions 117 may be arranged at the posterior end of the sensing array 108. These impedance sensing regions 117 may be configured to detect capacitance associated with proximity or contact with the human body to discern an adhesion state of the device on the garment 101.

Similarly, as discussed above, sensing array 108 may include one or more motion sensors 128 positioned throughout the sensing array 108. For example, a first motion sensor 128 may be positioned nearer to the anterior end of the sensing array and a second motion sensor 128 may be positioned nearer to the posterior end of the sensing array. These motion sensors may each include an IMU, accelerometer, gyroscope, magnetometer or a combination thereof.

Further, sensing array 108 may include one or more chemical sensors, such as sensors configured to detect gaseous compounds (or gas sensors). Sensing array 108 may include any number of digital gas sensors 120 or analog gas sensors 121, 131 as described above. The gas sensors may be positioned at predetermined locations on sensing array 108 such that when the sensing array 108 is positioned on the garment 101, the gas sensors are located in optimal positions to detect gases that may be expelled along with the excreta of interest, such as urine, feces or both.

With continued reference to FIG. 17, method 1700 may include, at step 1710, detecting, via the sensing array 108, an adhesion state of the sensing array to the garment 101. The adhesion state may indicate proper alignment of the sensing array 108 (within enclosure 104, in embodiments) with the garment 101. For example, the adhesion state may indicate the anterior portion of the sensing array 108 is aligned with the posterior area of the garment 101, that is to say, the sensing array 108 is positioned backwards or upside down, which would indicate a negative or improper adhesion state. Further, detecting the adhesion state comprises detecting at least one of a motion datum via the at least one motion sensor 128 or a capacitive datum via the at least one impedance sensing region 117.

For example, and without limitation, the adhesion state may indicate, based on the motion sensors 128 and/or the impedance sensing regions 117, proximity of the sensing array 108 to the human patient/user. The adhesion state therefore can imply when the device has been removed by the patient or has fallen off the garment 101. Further adhesion state may be detected by a combination of motion sensing and capacitive sensing. For example, and without limitation, the impedance sensing regions 117 may be denote when the sensing array 108 is at the intended distance from the user's body during intended performance times and motion detected by the motion sensors 128 may indicate the movement of the user over time, for example, if they have laid in the same position for an extended duration and need to be ambulated. In another example, if the motion sensors 128 detect movement of the sensing array 108 but the impedance sensing regions 117 detect close proximity to the human user, an indication that the sensing array 108 has slipped or has slid over the garment, perhaps due to the shifting of a patient in a hospital bed. Adhesion state may be recorded continuously, discrete or at the command of a medical provider or the patient themselves. Adhesion state may indicate movement data and proximity data separately, or simply return a binary recording that the sensing array is in the intended position at the intended times, or not. In various embodiments, the adhesion state may be recorded in one or more of a cloud database or electronic incontinence diary associated with the user. For example, and without limitation, the motion sensors 128 and impedance sensing regions 117 may update a variable in a cloud database as ‘true’ to indicate proper adhesion and alignment of sensing array 108 on garment 101.

With continued reference to FIG. 17, method 1700 may include, at step 1715, detecting, via the sensing array, an incontinence event within the garment. As described above, detecting an incontinence event within the garment 101 may include detection of one or both of urination and/or defecation. In various embodiments, detecting an incontinence event may include detecting at least one of an excreta presence, excreta location or excreta volume within the garment 101. Detecting excreta location may include detecting a thermal signature at one of the plurality of temperature sensors 112, such as a heat source presence in the garment 101 nearer to the anterior or front thereof, said detection may correspond to urination. Oppositely, detection of a heat source proximate the posterior or rear of the garment 101 may correspond to defecation. Detection of the incontinence event may include detection via one or more sensing modalities as described herein. For example, and without limitation, the impedance sensing regions 116a, 116b may detect urination proximate the anterior location of the garment 101 and detect defecation proximate the posterior of the garment. Further, the impedance sensing regions 116a, 116b and 118 may detect volume of excreta as volume of the liquid-based soil is related to a change in capacitance. Therefore, a linearization of the sensor readings may allow the user or a medical provider to discern the volume of excreta in the garment 101. Further, detecting the incontinence event may include detecting a chemical associated with one or both of urination and defecation in the form of gases expelled therewith. The gas sensors may be configured to each detect a different gaseous compounds or similar compounds. In other embodiments, gas sensors may be configured to detect the same gaseous compound or be placed within sensing array 108 proximate the area in which a known gaseous compound associated with an incontinence event is likely to appear. That is to say, for example, an ammonia sensor may be placed near the front of the sensing array 108 to detect urination by detecting the ammonia emanating therefrom.

When an incontinence event occurs, the CPU may perform math to determine the procession of an incontinence event. The CPU may then cast an alert to update the user interface with a notification stating that a patient has experienced an incontinence event. This alert may specify the type of incontinence event, which may inform different hygienic protocols to be taken. This alert may also specify the type of garment being worn by a patient, which may assist a caregiver or a nurse with acquiring the appropriate incontinence supplies for a given patient.

This alert may further specify the location of the patient needing incontinence care.

Any of the detections described throughout may be made relative to a predetermined threshold. For example, the sensing array 108 may automatically generate a temperature threshold above which the signals from the temperature sensors 112 would read as excreta presence. In the other sensing modalities, other thresholds may be set, manually or automatedly, such as capacitance, presence of gas (in parts per million (ppm), for example), position, velocity or acceleration in the case of the motion sensors, or the like.

A family member, nurse, caregiver, or care practitioner may then tend to a patient and administer incontinence care, which may including disposal of a soiled garment; cleaning of skin; gathering of clothing or laundry; bathing; application of any skin creams, powders, or ointments; or dressing with clean clothes and a clean garment. Upon completion of a change routine, said family member, nurse, caregiver, or care practitioner may then confirm the change routine's completion via an interactive icon on the user interface.

Battery management may occur as a part of the incontinence care routine, wherein a battery may be removed for charging; removed for disposal and replacement; or charged while embedded within a pad's architecture. Embedded batteries may be charged by direct cable connection or by a form of contactless charging, such as by induction. A notification of a low battery status may be given to inform appropriate battery management. Said management may occur with the coincidence of an incontinence event. Alternatively, said management may occur irrespective of the procession of an incontinence event.

With continued reference to FIG. 17, method 1700 may include, at step 1720, transmitting at least one signal from the sensing array 108 to at least one processor in communication with the sensing array 108. In various embodiments, the sensing array 108 may be in communication with a central processing unit (CPU) 132 configured to receive at least one signal from the sensing array 108 based on at least one sensor reading. The CPU 132 may be capable of reading sensor data, performing mathematical operations, and transmitting alerts regarding changes in device or patient status. In some cases, the CPU 132 may be placed within an electronics housing at one end of the sensing array 108.

The CPU 132 may receive signals from the sensing array 108 through various mechanisms. In some aspects, sensor interrupts may be employed to send alerts to the CPU 132 upon a sensor value exceeding, meeting, or falling below a certain threshold. Thresholds may be specified for each sensing method to determine cut-offs between traits of a clean garment and traits of a soiled garment. Alternatively, sensors may be continuously polled in a loop to be compared against some threshold or some result of a mathematical process.

In some cases, analog signals from the sensing array 108 may be converted to digital signals for processing by the CPU 132. Analog-to-digital converter (ADC) modules 152 may be connected to analog sensors to increase the resolution of analog signal readings. For example, analog gas sensors may be connected to ADC modules to augment gas signal quality. In some aspects, a capacitance-to-digital converter 137 may be employed to translate capacitive signals to digital signals.

In various embodiments a multiplexer may be included in the sensing array 108 to resolve address conflicts between repeated sensors. The multiplexer may also be used to reduce the current draw of the sensing array 108 by polling a portion of the sensors on the sensing array 108, rather than polling every sensor on said sensing array 108. This reduction in current draw may be favorable to reduce heat dissipation from the sensing array 108.

In various embodiments, the CPU 132 may include or be connected to a wireless communication module configured to transmit signals to a receiving unit. In some aspects, the CPU 132 may have a built-in or onboard antenna for wireless communication. In some cases, an external antenna 136 or an antenna module may be added to the CPU 132 to permit wireless communication. A communication protocol may be employed to transmit the values of the sensors to a receiving unit. The communication protocol may also be used to transmit the results of mathematical operations performed by the CPU 132 to the receiving unit.

When an incontinence event occurs, the CPU 132 may perform mathematical operations to determine the procession of an incontinence event. In various embodiments, as described below, the CPU or another processor may execute one or more machine learning models to classify sensor data to determine the occurrence of the incontinence event or a characteristic thereof, such as time, period, volume, location, or type, among others. CPU 132 may then cast an alert to update a user interface with a notification stating that a patient has experienced an incontinence event. This alert may specify the type of incontinence event, which may inform different hygienic protocols to be taken. The receiving unit may be a phone, tablet, laptop, desktop computer, or other electronic device configured to receive communications from the CPU 132 via wired or wireless protocols.

In various embodiments, method 1700 may include, at step 1725, generating, based on the incontinence event and the adhesion state, an incontinence diary associated with the user. The electronic patient incontinence diary may contain a log of a patient's incontinence events. The log may contain timestamps and dates for given events. For each event, the log may contain a classification of the type of incontinence event, being urinary, fecal, or dual in nature. In some cases, for each event, the log may contain a classification of the type of incontinence event with respect to an existing clinical continence assessment tool, where an example of such a tool may be the Bristol Stool Chart.

The electronic patient incontinence diary may be exported to an alternative file format, printed out, or shared digitally. A relevant party, such as a physician, a family, or an occupational therapist, may be the recipient of the electronic patient incontinence diary or reports derived therefrom.

In some aspects, the user interface may display a patient's incontinence status, wherein there may be a mode indicating a clean garment and a mode indicating a soiled garment. The user interface may also display a statement of pad adhesion, wherein there is at least a statement confirming the sensing array 108's adhesion to a patient's garment and a statement alerting to the sensing array 108 not being adhered to a patient's garment. In the scenario that an incontinence event has occurred, a statement of the time elapsed from when said event occurred may be displayed. A statement of estimated volume of urine or feces in a garment may also be displayed on the user interface.

The user interface that families, nurses, caregivers, and care practitioners may see could have different views depending on the party in question and may have multiple pages. For each patient on one page of the user interface, a patient's name, a related identifier, such as a patient's room number, or an anonymous identifier may be displayed. For each patient on this same page, a statement of a patient's incontinence status may be given, wherein there may be a mode indicating a clean garment and a mode indicating a soiled garment. For each patient on this same page, a statement of pad adhesion may be given, wherein there is at least a statement confirming a pad's adhesion to a patient's garment and a statement alerting to a pad not being adhered to a patient's garment. For each patient on this same page, an indicator of a pad's estimated battery level may be displayed. For each patient on this same page, an icon may be available to confirm a change was successfully completed. An icon may be available to reset a patient's displayed status, which may also be achieved with the aforementioned icon for change completion. In the scenario that an incontinence event has occurred, a statement of the time elapsed from when said event occurred may be displayed. Said statement of time elapsed may be interpreted as a statement of how long a patient has been exposed to a soiled garment. Said statement of time elapsed may be interpreted as a statement of the time taken for a patient's soiled garment to be changed. A statement of estimated volume of urine or feces in a garment may also be displayed.

Another page may be generated for each patient in specific. Said page may include a summary of a patient's information, which may include their name, their medical history, or the type of incontinence garment being worn. Said page may be in compliance with existing patient privacy laws or healthcare compliance regulations. Said page may have a summary of the current incontinence event if a patient has not been changed. Said summary may include a combination of a timestamp of when the event was detected; the type of incontinence event; or an estimate of the volume of soil in a garment. A section may exist for notes to be written by a nurse, family member, caregiver, or care practitioner. There may be an option to record instances of a patient denying care. Another unique result may also be generated for each patient: an electronic patient incontinence diary, wherein said diary contains a log of a patient's incontinence events. Said log may contain timestamps and dates for given events. For each event, said log may contain a classification of the type of incontinence event, being urinary, fecal, or dual in nature. For each event, said log may contain a classification of the type of incontinence event with respect to an existing clinical continence assessment tool, where an example of such a tool may be the Bristol Stool Chart. Said log may be exported to an alternative file format, printed out, or shared digitally. A relevant party, such as a physician, a family, or an occupational therapist, may be the recipient of said reports.

A device management page may be created to assist families and facilities who are operating multiple units of the incontinence monitoring pad. A patient management page may be created to assist families and facilities who have multiple patients using said units. An administrative page may be created to view metrics regarding incontinence care. Such metrics may include a measure of the time elapsed between incontinence events and successful change routines. Such metrics may also include statements of compliance between time elapsed in incontinence events and successful change routines to set markers, such as laws requiring checks on incontinence status. Such metrics may also include disease incidence rates or statements on the number of garments used. Such metrics may be visualized in a table, chart, graph, or other infographic modality. Such metrics may be summarized in reports. Said reports may be exported, printed out, or shared digitally. A relevant party, such as a government agency, an insurance company, a family, a physician, or an occupational therapist, may be the recipient of said reports. Decisions may be taken based on the data derived from said reports, including staffing, prioritization of certain incontinence care routines with respect to other patient care needs, or budgeting for appropriate response to incontinence care statuses.

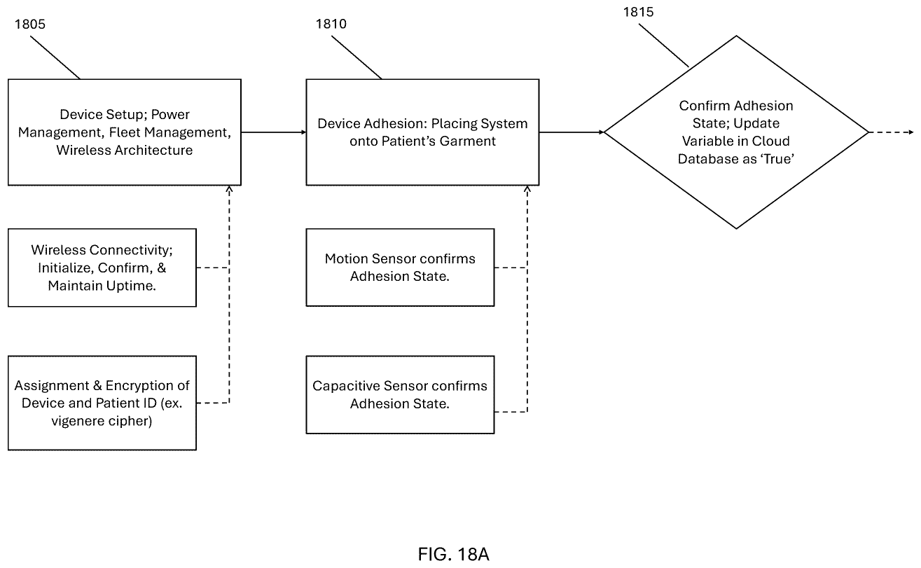

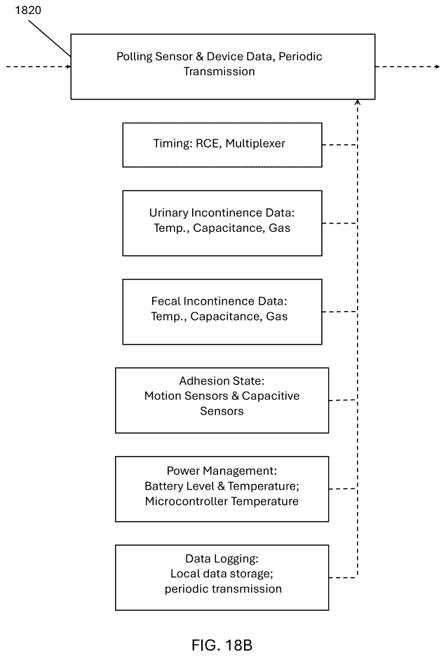

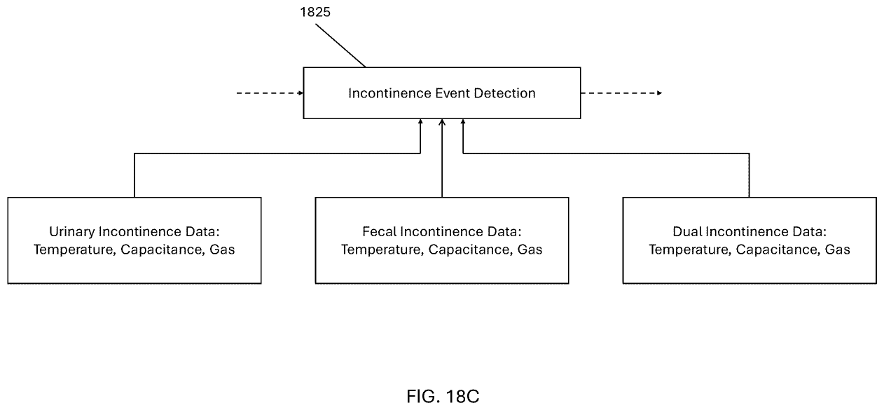

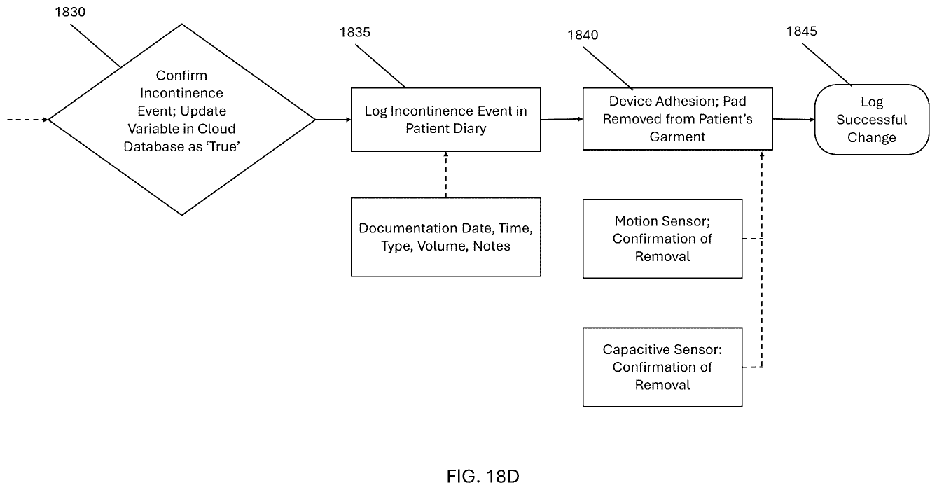

Referring now to FIGS. 18A-18D, a schematic representation of a method 1800 for monitoring incontinence events and adhesion states associated with the sensing array 108 is shown. As shown in FIG. 18A, method 1800 may include, at step 1805, performing device setup. Device setup may include power management, fleet management, and wireless architecture configuration. In various embodiments, device setup may include initializing wireless connectivity, confirming wireless connection, and maintaining uptime of the wireless communication module. In various embodiments, device setup may further include assignment and encryption of device and patient identifiers. For example, a cipher such as a Vigenère cipher may be employed to encrypt device and patient identifiers for secure communication and compliance with patient privacy laws or healthcare compliance regulations.

With continued reference to FIG. 18A, method 1800 may include, at step 1810, placing the system 100 onto a patient's garment 101 to establish device adhesion. As described above, the enclosure 104 containing the sensing array 108 may be adhered to the exterior of the garment 101 proximate the absorbent region 102. In various embodiments, the motion sensors 128 may confirm the adhesion state by detecting rotation and translation associated with the sensing array 108 being attached to the garment 101. In various embodiments, the capacitive sensing regions 117 may confirm the adhesion state by registering a high signal when in proximity with a human body, owing to the large capacitance of the human body. The combination of motion sensor data and capacitive sensor data may be used to confirm the adhesion state of the sensing array 108 to the garment 101.

As shown in FIG. 18A, method 1800 may include, at step 1815, confirming the adhesion state and updating a variable in a cloud database to indicate that the sensing array 108 is adhered to the patient's garment 101. Once the adhesion state is confirmed, the method 1800 may proceed to polling sensor and device data as shown in FIG. 18B.

Referring now to FIG. 18B, method 1800 may include, at step 1820, polling sensor and device data with periodic transmission to a receiving unit. In various embodiments, a timing element 148, such as a real time clock (RTC), and a multiplexer 144 may be employed to coordinate the polling of sensors on the sensing array 108. The multiplexer 144 may be used to reduce the current draw of the sensing array 108 by polling a portion of the sensors rather than polling every sensor simultaneously. Urinary incontinence data, including temperature data from the plurality of temperature sensors 112, capacitance data from the impedance sensing regions 116, and gas data from the gas sensors 120, 121, may be collected during the polling process.

With continued reference to FIG. 18B, a detailed view of the polling sensor and device data process is shown. In various embodiments, the polling process may include collecting timing data via the timing element 148 and multiplexer 144. Urinary incontinence data, including temperature, capacitance, and gas readings from sensors proximate the anterior surface of the user, may be collected. Fecal incontinence data, including temperature, capacitance, and gas readings from sensors proximate the posterior surface of the user, may be collected. Adhesion state data from the motion sensors 128 and capacitive sensing regions 117 may be monitored to ensure the sensing array 108 remains properly adhered to the garment 101.

With continued reference to FIG. 18B, the polling process may further include power management monitoring. In various embodiments, the battery 140 level and temperature may be monitored, as well as the temperature of the CPU 132. A sensing component, such as a thermistor or temperature sensor, may be connected to the CPU 132 to monitor its temperature and issue an alert or interrupt upon the CPU temperature exceeding a threshold temperature. Data logging may be performed via the memory storage module 160, which may provide local data storage and facilitate periodic transmission of sensor data to a receiving unit via the wireless communication module.

With continued reference to FIG. 18C, method 1800 may include, at step 1825, performing incontinence event detection based on the polled sensor data. Urinary incontinence data and fecal incontinence data, each comprising temperature, capacitance, and gas sensor readings, may be analyzed to detect the occurrence of an incontinence event. The method 1800 may then proceed to classify the incontinence event as shown in FIG. 18D.

Referring now to FIG. 18D, a schematic representation of incontinence event detection classification is shown. The incontinence event detection process may receive sensor data and classify the incontinence event based on the type of excreta detected. In various embodiments, urinary incontinence data comprising temperature, capacitance, and gas sensor readings may be analyzed to detect urinary incontinence events. Fecal incontinence data comprising temperature, capacitance, and gas sensor readings may be analyzed to detect fecal incontinence events. Dual incontinence data comprising temperature, capacitance, and gas sensor readings from both the anterior and posterior regions may be analyzed to detect dual incontinence events in which both urinary and fecal incontinence occur. The classification of the incontinence event type may inform different hygienic protocols to be taken by a caregiver or nurse.

With continued reference to FIG. 18D, method 1800 may include at step 1830 confirming incontinence event in one or more database, such as a cloud database, as ‘true’.

Further, at 1835 the time, date, type, volume and any notation regarding the incontinence event in a patient incontinence diary. Further, at step 1840, the incontinence monitoring system may be removed from a patient's garment. Motion sensors and capacitive sensors as described herein and carried on the sensing array within the monitoring system may confirm device removal. At 1845, the system may log a successful garment or monitoring system change associated with event detection, diary entry and removal of the system or garment from the patient.

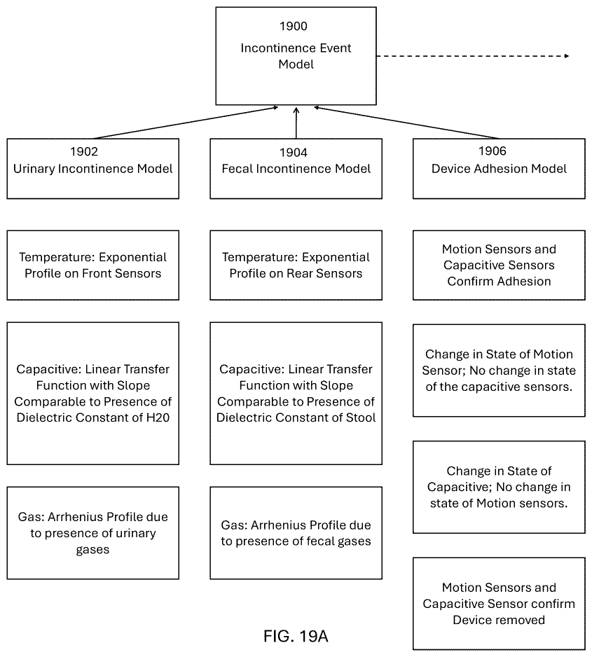

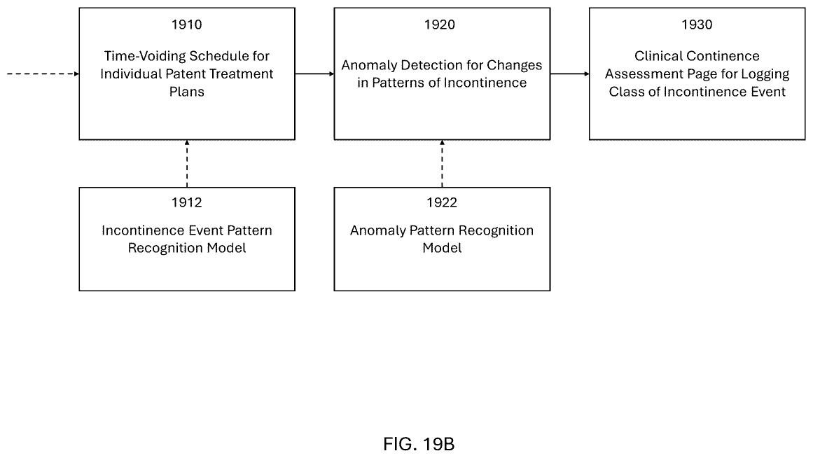

Referring now to FIG. 19A-19B, a schematic representation of an incontinence event machine learning model 1900 (incontinence model 1900) is shown. Incontinence model 1900 may be executed by one or more processors in communication with or carried on sensing array 108. For example, CPU 132 may execute incontinence model 1900. Incontinence model 1900 may be include or be in communication with a separate machine learning model designated for each incontinence event type and adhesion state. As shown, incontinence model 1900 may include urinary incontinence model 1902, fecal incontinence model 1904 and device adhesion model 1906. Each model may be configured to classify sensor profiles associated with detection of a given event, such as urinary incontinence event, fecal incontinence event and adhesion state of the sensing array 108 on the garment 101. As shown in FIG. 19A, by way of example, urinary incontinence model 1902 may be configured to classify sensor profiles associated with detecting urinary incontinent events, such as the plurality of temperature sensors 112 proximate the anterior surface of the user, transfer functions of the capacitive readings comparable to presence of water and the Arrhenius gas profile due to the presence of urinary gases as described above.

Similarly, by way of example, fecal incontinence model 1904 may be configured to classify sensor profiles associated with detecting fecal incontinent events, such as the plurality of temperature sensors 112 proximate the posterior surface of the user, transfer functions of the capacitive readings comparable to presence of stool and the Arrhenius gas profile due to the presence of fecal gases as described above. Further, again by way of example, adhesion model 1906 may be configured to classify sensor profiles associated with detecting the adhesion state, such as the readings from the motion sensors 128, capacitive sensing regions 117, and CPU interpretation of a change in state of either motion of capacitive sensors corresponding to device adhesion or removal.

Machine learning may be employed to perform classification of sensor profiles by comparing a patient's sensor data with a trained model on sensor data for an incontinence event. One or more of the above-described models may be trained on a patient's incontinence event history or continuously trained as incontinence events are detected and input into an electronic incontinence diary of a patient. Alternatively, machine learning may be employed to perform curve-fitting or regression analysis to compare a patient's sensor data with a trained model on features of incontinence for a given sensing method. Identification of incontinence event waveforms may alternatively be achieved with convolutional neural networks, recurrent neural networks, or long short-term memory networks, among others which may be established to discern key characteristics of incontinence events and adhesion state.

With continued reference to FIGS. 19A-19B, a time-voiding schedule 1910 may be generated collecting a patient's incontinence event history for a duration of time, such as the data associated with training the aforementioned models, wherein the length of said duration may vary or may be determined by a treatment plan. Said treatment plan may be established by a medical practitioner or a caregiver. In various embodiments, one or more algorithms, or further ML models, shown in FIG. 19B incontinence event pattern recognition model 1912 can be executed to process said event history and identify patterns of incontinence. For example, and without limitation, features such as the number of events logged per day, can be provided or retrieved by pattern recognition model 1912 can perform a descriptive statistical analysis on the average frequency of daily incontinence events. Another feature that may be examined includes the types of incontinence events that occur per day, where the type refers to the nature of said event being urinary, fecal, or dual incontinence. Another feature that may be examined includes the time of day when events occur, which may further extrapolate into identifying times of day when a certain type of event transpires. A unique result can then be generated as a notification, schedule, agenda, or a table to recommend to a family, nurse, caregiver, or care practitioner when a patient may typically experience the one or more incontinence events based on their longitudinal history. Another unique result that may be generated is a notification alerting a family, nurse, caregiver, or care practitioner to any deviance from a patient's long-term incontinence behavior, which may be characterized based on changes in frequency of events as determined at the anomaly detection block 1920 by anomaly recognition model 1922, such as changes in the types of events, changes in the timing of events throughout a given day, or other features. Said schedule, agenda, or table may be exported to an alternative file format, printed out, or shared digitally. A relevant party, such as a physician, a family, or an occupational therapist, may be the recipient of said reports at a clinical incontinence assessment page, represented by block 1930.

Any system or method described herein may be trained via incontinence event simulation. Further, in various embodiments, incontinence simulation may be used to test the herein disclosed systems and methods. In various implementations, a method of simulating incontinence may include an architecture built to imitate human anatomy for the urinary and fecal excretion pathways. Said architecture may contain two principal orifices: an orifice intending to expel urine or an analogous mixture to urine; and an orifice intending to expel feces or an analogous mixture to feces. Tubing may be used to guide analogous waste through said orifices. A garment may be dressed over this architecture, with the absorbent region of said garment arranged proximate the simulated orifices. In various embodiments, samples of urine, feces, blood, saliva, serum, semen, other bodily fluids or excreta, or analogous compounds may be inserted into the architecture's tubing and then released through the designated orifices. Said architecture may be fabricated by subtracting material from an existing anatomical model or human mannequin. Alternatively, said architecture can be made by an additive manufacturing method to form a figure resembling human anatomy.

In various embodiments, said architecture may include the use of valves to simulate different forms of incontinence, such as incomplete voiding or constipation. In various embodiments, said architecture may further include a means of actuating said valves to imitate precise physiological states. Improved versions of said architecture may include flow meters to offer numerical estimates for flow of materials through the architecture.

Referring now to FIGS. 20A-20C, schematic views of embodiments of an incontinence monitoring systems 2001, 2002 and 2003 are shown in perspective views. In various embodiments, the systems shown herein may include a sensing array 108 as described above. One of skill in the art would appreciate that only an illustrative schematic of the centerline-arranged temperature sensors are shown disposed along the sensing array 108 from the first end to the second end, but any number of sensors described herein may be present on sensing array 108.

Referring specifically to FIG. 20A, incontinence monitoring system 2001 may include sensing element 108 configured to span the area of a human patient proximate the urethra and the anus. Sensing element 108 may include fastening elements 2005 disposed at the first end and the second end. The fastening elements 2005 may correspond to fastening elements 2010 disposed on opposite and opposing T-shaped coupling elements 2004. The T-shaped coupling elements 2004 may include a stem portion aligned with the central axis of the sensing element 108 and transverse end portions disposed perpendicular to the stem portions. The stem portions may intersect the transverse end portions and proximate the midpoint thereof, respectively. The sensing array-side fastening elements 2005 may be configured to engage with and releasably attached to the T-side fastening elements 2010. Accordingly, the sensing array 108 may be suspended between the two T-shaped coupling elements 2004. The T-shaped coupling portions may then attach directly to garment 101 in order to position the sensing array 108 in the proper orientation to detect one or more incontinence events experienced by a patient. In various embodiments, each transverse T-shaped end portion may also circumscribe the trunk of a patient proximate the waist area to secure sensing array 108 without the need for an enclosure 104 or garment 101 necessarily.

Referring now to FIG. 20B, an incontinence monitoring system 2002 with a similar arrangement of sensing array 108. In system 2002, the sensing array 108 may be releasably coupled to coupling element 2012, which in this case, is a continuous element having two opposite transvers T-shaped portions connected by a central longitudinal element which is longer than sensing array 108. Similar to FIG. 20A, sensing array 108 may engage and releasably couple to coupling element 2012 via corresponding array-side fastening elements 2005 and T-side fastening elements 2010. The central longitudinal portion may support the sensing array 108 from underneath, opposite the sensor-side of the sensing array 108. The T-shaped coupling portions may then attach directly to garment 101 in order to position the sensing array 108 in the proper orientation to detect one or more incontinence events experienced by a patient. In various embodiments, each transverse T-shaped end portion may also circumscribe the trunk of a patient proximate the waist area to secure sensing array 108 without the need for an enclosure 104 or garment 101 necessarily.

Referring now to FIG. 20C, an incontinence monitoring system 2003 is shown. In this embodiment, the incontinence monitoring system 2003 may include a T-shaped sensing array 2014. The T-shaped sensing array 2014 may be substantially similar to sensing array 108 and include any sensors as described throughout the disclosure. T-shaped sensing array 2014 may include longitudinally positioned temperature sensors 112, however any other types of sensors may be positioned here as well or instead. T-shaped sensing array 2014 may include sensors on the transverse end portion, such as digital gas sensors 120 and analog gas sensors 121, 131 disposed thereon for optimal placement when T-shaped sensing array 2014 is coupled to the opposite T-shaped coupling element 2016, shown in broken lines. When connected together, the incontinence monitoring system 2003 may circumscribe the waist of a patient to secure the T-shaped sensing array 2014 properly on the patient to detect incontinence events. In various embodiments, the T-shaped end portions of both the sensing array 2014 and coupling element 2016 may additionally or alternatively couple to garment 101.

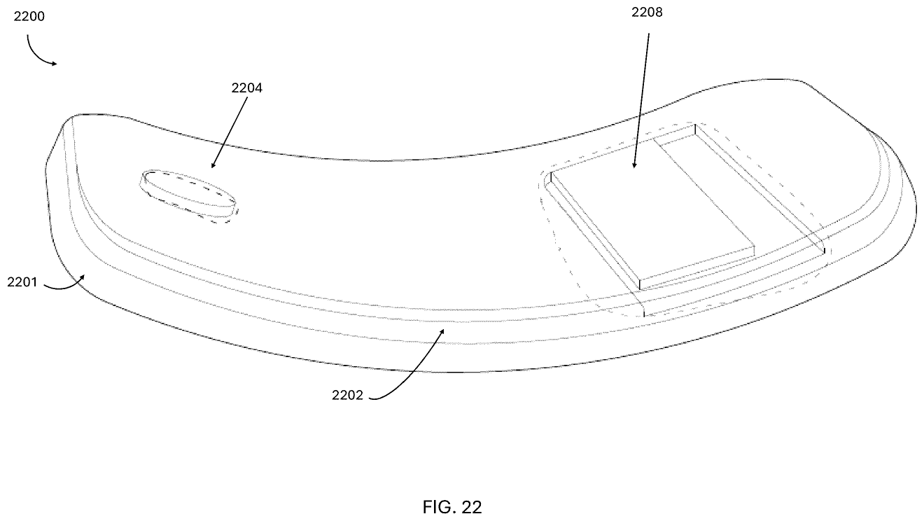

Referring now to FIG. 22, another embodiment of an incontinence system 2200 is shown. System 2200 may include an enclosure formed by an upper layer 2202 and a lower layer 2201. In various embodiments, the upper layer 2202 and lower layer 2201 may be attached along their overlapping perimeters to define an interior cavity, similar to the enclosure 104 described above with reference to FIGS. 3A-3B.

System 2200 may further include an anterior sensing area 2204 configured to detect urine. In various embodiments, the anterior sensing area 2204 may be positioned proximate the front of the system 2200 such that when worn on a garment, the anterior sensing area 2204 is disposed proximate the urethra of the user. The anterior sensing area 2204 may include one or more of the sensors described throughout this application. For example, and without limitation, the anterior sensing area 2204 may include one or more of the plurality of temperature sensors 112, one or more of the impedance sensing regions 116 (such as capacitive sensing regions 116a, 116b), one or more gas sensors (such as digital gas sensors 120, analog gas sensors 121, 131), and one or more overlay elements 113 positioned over the temperature sensors 112.

System 2200 may further include a posterior detection area 2208 configured to detect feces. In various embodiments, the posterior detection area 2208 may be positioned proximate the rear of the system 2200 such that when worn on a garment, the posterior detection area 2208 is disposed proximate the anus of the user. The posterior detection area 2208 may similarly include one or more of the sensors described throughout this application. For example, and without limitation, the posterior detection area 2208 may include one or more of the plurality of temperature sensors 112, one or more of the impedance sensing regions 116 (such as capacitive sensing regions 116a, 116b), one or more gas sensors (such as digital gas sensors 120, analog gas sensors 121, 131), and one or more overlay elements 113 positioned over the temperature sensors 112.

In various embodiments, each of the anterior sensing area 2204 and the posterior detection area 2208 may include any combination of the sensors described throughout this application. For example, each sensing area may include one or more motion sensors 128 for detecting adhesion state, one or more sensing regions 117 for detecting proximity or contact with a human body, and a CPU 132 in communication with the sensors for processing sensor data and transmitting alerts regarding incontinence events or adhesion states. In some embodiments, the anterior sensing area 2204 and the posterior detection area 2208 may share common components, such as a single CPU 132, ground bus 118, or power source. In various other embodiments, each sensing area may include dedicated components for independent operation.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. A system for monitoring incontinence, the system comprising:
a garment wearable by a user, the garment comprising an absorbent region;
a selectively openable enclosure defining a cavity, the enclosure configured to adhere to the garment proximate the absorbent region and along a sagittal plane of the user;
a sensing array positioned within the cavity of the enclosure, the sensing array having a first end proximate an anterior surface of the user and a second end proximate a posterior surface of the user, the sensing array comprising:
a plurality of temperature sensors arranged linearly between the first end and the second end;
at least one impedance sensing region;
at least one motion sensor positioned proximate the first end or the second end; and
at least one gas sensor positioned proximate the first end or the second end.

2. The system of claim 1, comprising an overlay element arranged to contact at least a portion of the plurality of temperature sensors, the overlay element comprising a conductive material.

3. The system of claim 2, wherein the conductive material comprises at least one of copper or silver.

4. The system of claim 1, wherein the at least one impedance sensing region comprises a first capacitive sensing region positioned proximate the first end and a second capacitive sensing region positioned proximate the second end of the sensing array.

5. The system of claim 1, wherein the at least one gas sensor comprises at least a first gas sensor proximate the first end and at least a second gas sensor proximate the second end.

6. The system of claim 5, wherein the first and second gas sensors are each configured to detect different gaseous compounds associated with urination and defecation, respectively.

7. The system of claim 5, wherein the first gas sensor is configured to detect at least one of ammonia (NH3), dihydrogen sulfide (H2S), odor or volatile organic compounds (VOC); and
the second gas sensor is configured to detect at least one of methane (CH4), dihydrogen sulfide (H2S), odor or volatile organic compounds (VOC).

8. The system of claim 1, wherein the at least one motion sensor comprises an inertial measurement unit (IMU), the IMU comprising at least one of an accelerometer, a gyroscope or a magnetometer.

9. The system of claim 1, wherein the sensing array is communicatively coupled to at least one processor.

10. The system of claim 1, wherein the enclosure comprises a coupling element extending longitudinally along an exterior surface, each end of the coupling element extending beyond the ends of the enclosure, the ends of the coupling element configured to couple to the garment.

11. A method for monitoring incontinence, the method comprising:
positioning a sensing array along an absorbent region of a garment, the sensing array arranged substantially along an axis aligned with a sagittal plane of a user wearing the garment, the sensing array comprising:
a first end proximate an anterior surface of the user and a second end proximate a posterior surface of the user;
a plurality of temperature sensors arranged coaxially with the axis between the first end and the second end;
at least one impedance sensing region;
at least one gas sensor; and
at least one motion sensor;
detecting, via the sensing array, an adhesion state of the sensing array to the garment;
detecting, via the sensing array, an incontinence event within the garment; and
transmitting at least one signal from the sensing array to at least one processor in communication with the sensing array.

12. The method of claim 11, further comprising retrieving an incontinence event history associated with the user prior to the positioning of the sensing array.

13. The method of claim 11, further comprising generating, based on the adhesion state and the incontinence event, an incontinence diary associated with the user.

14. The method of claim 11, wherein detecting the adhesion state comprises detecting at least one of a motion datum via the at least one motion sensor or a capacitive datum via the at least one impedance sensing region.

15. The method of claim 11, wherein detecting the incontinence event comprises detecting at least one of an excreta presence, an excreta location, or an excreta volume within the garment.

16. The method of claim 15, wherein detecting the excreta location comprises at least one of detecting a gaseous compound via the at least one gas sensor or detecting a temperature gradient across the plurality of temperature sensors.

17. The method of claim 15, wherein detecting the excreta volume comprises detecting a change in capacitance via the at least one impedance sensing region.

18. The method of claim 15, wherein detecting the excreta presence comprises detecting a temperature at at least one temperature sensor above a predetermined temperature threshold associated with a clean garment.

19. The method of claim 12, further comprising:
training a machine learning model on at least the incontinence event history associated with the user; and
executing, by the at least one processor, the machine learning model, to classify the signals for each sensor of the sensing array.

20. The method of claim 19, wherein the machine learning model is at least one of a convolutional neural network (CNN), recurrent neural network (RNN) or a long short-term memory network.
